(19) United States
(12) Patent Application Publication
(10) Pub. No.: US 2003/0026762 A1
(43) Pub. Date: Feb. 6, 2003

(54) Bio-spectral imaging system and methods for diagnosing cell disease state

(76) Inventors: Mark K. Malmros, Washington Crossing, PA (US); William S. Collins II, San Diego, CA (US); Pier J. Cipriani, Newtown, PA (US); Dennis M. Loreman JR., Bristol, PA (US)

Correspondence Address: Henry N. Wixon, Hale and Dorr LLP, 1455 Pennsylvania Avenue N.W., Washington, DC, 20004, US

(21) Appl. No.: 10/132,278

(22) Filed: Apr. 26, 2002

Related U.S. Application Data
(63) Continuation-in-part of application No. 09/306,662, filed on May 5, 1999.

(51) Int. Cl. A61K 49/00

(52) U.S. Cl. 424/9.6; 514/224.8

(57) ABSTRACT

A bio-spectral imaging system and methods for in situ diagnosis of a disease state in biological tissue or cells of a living organism are provided. In one form, a method includes applying to the tissue or cells in situ a biological stain composition that includes at least one metachromatic dye; performing a spectral analysis to obtain at least one spectrum of the stained tissue or cells; comparing the spectrum with spectra from a library of spectra of similarly stained reference tissue or cells that have been categorized as diseased or non-diseased; and correlating the spectrum to a disease state. The system and methods may be used to diagnose, for example, pre-cancer and/or cancer, as well as diseases caused by various microorganisms. Methods for creating a library of spectra, methods for monitoring the efficiency of photodynamic therapy of diseased biological tissue or cells in vivo, as well as methods for improving the efficiency of photodynamic therapy are also provided.

The present application is a continuation-in-part of co-pending U.S. patent application Ser. No. 09/306,662, filed May 5, 1999, and is hereby incorporated by reference in its entirety.

### BACKGROUND OF THE INVENTION

The present invention relates generally to a bio-spectral imaging system and methods for diagnosing a disease state in biological tissue or cells of a living organism. Specifically, the invention relates to a bio-spectral imaging system and methods for diagnosing a disease state in biological tissue or cells of a living organism utilizing biological stain compositions and spectroscopic methods.

Detection of a disease state is a necessary first step in treating the disease. The earlier the disease is detected, the earlier treatment may begin, leading to a better prognosis. For example, the early recognition of abnormal tissues, such as dysplastic, pre-cancerous or cancerous tissues, is of primary importance in eradicating, or otherwise decreasing the effects of, such a disease state. A wide variety of techniques are known to detect various disease states.

For example, histological examinations are known to diagnose cell disease states. A reliable histopathological diagnosis is an indispensable precondition of the successful treatment of a variety of diseases and disorders. However, such methods require a trained individual to examine the tissue or cells in each case of a suspected disease state.

Spectroscopic methods are also known to diagnose various disease states. Many of such methods require complex, expensive machinery. Additionally, such methods are limited in the variety of disease states that may be diagnosed. A need therefore exists for a bio-spectral imaging system and methods of diagnosing disease states. The present invention addresses this need.

### SUMMARY OF THE INVENTION

It has been discovered that the extent of metachromasia determined in biological tissues and/or cells stained with a metachromatic dye may be correlated to a disease state. Accordingly, methods for in situ diagnosis of a disease state in biological tissue or cells of a living organism are provided, as are methods for monitoring the efficiency of photodynamic therapy and methods for increasing the efficiency of photodynamic therapy. Methods of creating a library of spectra of biological tissue or cells of a living organism are also provided, as is a bio-spectral imaging system that may be advantageously used, for example, in the methods described herein.

In one form of the invention, a method includes applying to test tissue or cells in situ a biological stain composition to form stained test tissue or cells. In certain embodiments, the biological stain composition includes at least one metachromatic dye. The dye is further preferably a thiazine dye, such as methylene blue or toluidine blue 0. A spectral analysis of the stained test tissue or cells is performed to obtain at least one spectrum, preferably a reflected light spectrum. The spectrum from the stained test tissue or cells is compared with the spectra from a previously created library of spectra of similarly stained reference tissue or cells that have been categorized as diseased or non-diseased, preferably by a histological technique.

In certain forms of the invention, the spectral analysis includes determining the degree of the metachromatic shift of the dye from the spectrum, such as a reflected light spectrum, of the stained test tissue or cells. Additionally, in other forms of the invention, comparing the spectrum from the stained test tissue or cells with the spectra from the library of spectra of the reference tissue or cells includes comparing the degree of the metachromatic shift of the dye from the reflected light spectrum of the stained test tissue or cells with the degree of the metachromatic shift of the dye from the reflected light spectrum of the dye from the library of previously obtained spectra of similarly stained reference tissue or cells. The spectrum of the stained test tissue or cells may then be correlated with a disease state, so that an in situ diagnosis of a disease state may be made.

In further aspects of the invention, methods of creating a library of spectra of biological tissue or cells of a living organism are provided. In one form, a biological stain composition including at least one metachromatic dye is applied to tissue or cells to form stained tissue or cells. It is then determined whether the stained tissue or cells are diseased or non-diseased, preferably by performing a histological assay. A spectral analysis of the stained tissue or cells is performed to obtain at least one spectra. The spectral features of the spectrum are then analyzed. In one form of the invention, the analysis includes determining the degree of the metachromatic shift of the dye from the spectrum. The spectral features of the spectrum may then be correlated to a disease state.

In other aspects of the invention, methods for monitoring the efficiency of photodynamic therapy of diseased biological tissue or cells in vivo are provided. In one form, a method includes applying to the tissue or cells a photosensitizing dye, such as a metachromatic dye, to form stained tissue or cells. A spectral analysis is then performed on the stained test tissue or cells to obtain at least one spectrum, preferably a reflected light spectrum. The spectrum from the stained test tissue or cells is compared with the spectra from a previously created library of spectra of similarly stained reference tissue or cells that have been categorized as diseased or non-diseased, preferably by a histological technique. The stained test tissue or cells is then irradiated with light having an intensity sufficient to induce photooxidation of the dye. Changes in the spectrum of the stained test tissue or cells are monitored by the spectral analysis described above. In preferred forms of the invention, changes in the degree of the metachromatic shift of the dye are monitored.

In preferred forms of a method for monitoring the efficiency of photodynamic therapy of diseased biological tissue or cells in vivo, the spectral analysis includes determining the degree of the metachromatic shift of the dye from the spectrum, such as a reflected light spectrum, of the stained test tissue or cells. Additionally, in other forms of the invention, comparing the spectrum from the stained test tissue or cells with the spectra from the library of spectra of the similarly stained reference tissue or cells includes comparing the degree of the metachromatic shift of the dye from the reflected light spectrum of the stained test tissue or cells with the degree of the metachromatic shift of the dye from the reflected light spectrum of the dye from the library of previously obtained spectra of similarly stained reference tissue or cells.

In a further aspect of the invention, methods for improving the efficiency of photodynamic therapy of diseased tissue or cells in vivo are provided. In one form, a method includes applying to the tissue or cells a photosensitizing dye, such as a metachromatic dye, to form stained tissue or cells. A spectral analysis is then performed on the stained test tissue or cells to obtain at least one spectrum, preferably a reflected light spectrum. The spectrum from the stained test tissue or cells is compared with the spectra from a previously created library of spectra of similarly stained reference tissue or cells that have been categorized as diseased or non-diseased, preferably by a histological technique. The stained test tissue or cells is then irradiated with light having an intensity sufficient to induce photooxidation of the dye. Changes in the spectrum of the stained test tissue or cells are monitored by the spectral analysis described above. In preferred forms of the invention, changes in the degree of the metachromatic shift of the dye are monitored.

In preferred forms of a method for improving the efficiency of photodynamic therapy of diseased biological tissue or cells in vivo, the spectral analysis includes determining the degree of the metachromatic shift of the dye from the spectrum, such as a reflected light spectrum, of the stained test tissue or cells. Additionally, in other forms of the invention, comparing the spectrum from the stained test tissue or cells with the spectra from the library of spectra of the similarly stained reference tissue or cells includes comparing the degree of the metachromatic shift of the dye from the reflected light spectrum of the stained test tissue or cells with the degree of the metachromatic shift of the dye from the reflected light spectrum of the dye from the library of previously obtained spectra of similarly stained reference tissue or cells.

Additionally, the method of increasing the efficiency of photodynamic therapy of diseased tissue or cells in vivo includes adjusting the intensity of the irradiating light or the duration of the therapy, repositioning the source of the irradiating light, or a combination thereof, based on the changes in the spectrum of the stained test tissue or cells. In preferred forms of the invention, the intensity of the irradiating light or the duration of the therapy is adjusted, or the source of the irradiating light is repositioned, or a combination thereof, based on the difference between the degree of the metachromatic shift of the dye from the reflected light spectrum of the stained tissue or cells with the degree of the metachromatic shift of the dye from the reflected light spectrum of the similarly stained reference tissue or cells.

In yet other aspects of the invention, a bio-spectral imaging system is provided. The imaging system may be advantageously used, for example, in the methods of the present invention. An exemplary imaging system in accordance with the present invention includes at least one, and preferably two or more light sources having a substantially same first wavelength. The system also includes at least one, and preferably two or more light sources having a substantially same second wavelength, the second wavelength being different than the first wavelength. An imaging plane, adapted to support a plurality of stained cells positioned adjacent to the imaging plane, receives sequential illumination from at least one, and preferably from at least two, light source(s) at the respective first and second wavelengths. A plurality of photoreceptors, arranged for movement relative to the imaging plane, is positioned to digitally record an image of at least a portion of the stained cells, during each of the sequential illuminations of the cells at the first and second wavelengths. In another embodiment, the imaging plane can optionally move relative to the plurality of photoreceptors.

The data captured by the system can be processed by a general purpose computer using, for example, intensity ratios of light reflected from the stained cells at the various wavelengths. In accordance with at least one algorithm, the cells can be categorized as being abnormal when the stain reflects no more light at the second wavelength, approximately 30 nanometers less that the first wavelength, than at the first wavelength at which minimum reflection of light occurs in normal cells. When methylene blue is used as a stain, the first wavelength is approximately 665 nanometers.

In at least one embodiment, the light sources are concentrically spaced around the plurality of photoreceptors, and have an angle of incidence of approximately 45 degrees with respect to the imaging plane. Further, when two or more light sources are used at each wavelength, each of the light sources at a given wavelength are spaced substantially equidistant with respect to each other. Finally, a holographic diffuser can be used in conjunction with each light source.

It is an object of the invention to provide methods for in situ diagnosis of a disease state in biological tissue or cells of a living organism.

It is another object of the invention to provide methods of creating a library of spectra of biological tissue or cells of a living organism.

It is a further object of the invention to provide methods for monitoring the efficiency of photodynamic therapy of diseased biological tissue or cells.

It is yet another object of the invention to provide methods for increasing the efficiency of photodynamic therapy.

It is a further object of the invention to provide a bio-spectral imaging system. the imaging system may be used in the methods described herein.

These and other objects and advantages of the present invention will be apparent from the descriptions herein.

### BRIEF DESCRIPTION OF THE FIGURES

FIG. 1 depicts an RGB composite image of normal nasal tissue stained and analyzed as described in Example 1.

FIG. 2 shows a spectral graph of reflected light intensity as a function of wavelength for three regions of interest in the RGB composite image of FIG. 1.

FIG. 3 depicts an RGB composite image of normal tissue stained and analyzed as described in Example 1.

FIG. 4 depicts a spectral graph of reflected light intensity as a function of wavelength for three regions of interest in the RGB composite image of FIG. 3.

FIG. 5 shows an RGB composite image of tissue stained and analyzed as described in Example 1 and showing small regions of basal cell carcinoma with associated inflammation.

FIG. 6 depicts a spectral graph of reflected light intensity as a function of wavelength for three regions of interest in the RGB composite image of FIG. 5.

FIG. 7 depicts an RGB composite image of tissue stained and analyzed as described in Example 2.

FIG. 8 depicts a spectral graph of reflected light intensity as a function of wavelength for the five regions of interest in the RGB composite image of FIG. 7.

FIG. 9 depicts an RGB composite image of the top region of the tissue of FIG. 7 stained and analyzed as described in Example 2.

FIG. 10 depicts a spectral graph of reflected light intensity as a function of wavelength for the five regions of interest in the RGB composite image of FIG. 9.

FIG. 11 shows a spectral map of the tissue sample of FIG. 5 that has been stained and analyzed as described in Example 3.

FIG. 12 shows a spectral map of a cheek tissue sample stained and analyzed as described in Example 3. The image is mapped in dark blue corresponding to those voxels matching MI 45.

FIG. 13 depicts a spectral map of a cheek tissue sample of FIG. 12 that is mapped in light blue corresponding to those voxels matching MI 45 and MI 01.

FIG. 14 depicts a spectral map of a tissue sample from the helix of the ear (i.e., from the external prominent rim of the ear) stained and analyzed as described in Example 3. The image is mapped in dark blue corresponding to those voxels matching MI 45.

FIG. 15 depicts the spectral map of the tissue sample in FIG. 14, mapped in light blue together with the MI 45 map and an MI Class 2 map.

FIG. 16 shows the spectral map of a pre-auricular (i.e., an area of the outer part of the ear just below the rim) tissue sample stained and analyzed as described in Example 3. The image is mapped in dark blue corresponding to those voxels matching MI 45.

FIG. 17 depicts the spectral map of the tissue sample of FIG. 16, mapped in light blue together with the MI 45 map and an MI Class 1 map.

FIG. 18 depicts the spectral map of a finger tissue sample stained and analyzed as described in Example 3. The image is mapped in dark blue corresponding to those voxels matching MI 45.

FIG. 19 depicts the spectral map of the tissue sample of FIG. 18, mapped in light blue together with the MI 45 map and an MI Class 2 map.

FIG. 20 depicts the spectral map of a nasal tissue sample stained and analyzed as described in Example 3. The image is mapped in dark blue corresponding to those voxels matching MI 45.

FIG. 21 depicts the spectral map of the tissue sample of FIG. 20, mapped in light blue together with the MI 45 map and an MI Class 2 map.

FIG. 22 depicts a spectral graph of reflected light intensity as a function of wavelength for three regions of interest in the RGB composite image of FIG. 23.

FIG. 23 depicts an RGB composite image of a scalp tissue sample stained and analyzed as described in Example 4.

FIG. 24 shows a spectral map of a scalp tissue sample stained with toluidine blue O and analyzed as described in Example 5. The image is mapped in dark blue corresponding to those voxels matching MI 01 where the relative intensity at 600 nanometers (nm) is greater than at 610 nm.

FIG. 25 depicts a spectral map of the tissue sample of FIG. 24, mapped in light blue corresponding to those voxels matching MI 01 where the relative absorbance at 600 nm/610 nm absorption intensity is greater than at 640 nm.

FIG. 26 depicts a spectral map of a temple/ear tissue sample stained with toluidine blue O and analyzed as described in Example 5. The image is mapped in dark blue corresponding to those voxels matching MI 01 where the relative intensity at 600 nm is greater than at 610 nm.

FIG. 27 depicts a spectral map of the tissue sample of FIG. 26, mapped in light blue corresponding to those voxels matching MI 01 where the relative absorbance at 600 nm/610 nm absorption intensity is greater than at 640 nm.

FIG. 28 is an exemplary elevation view of the system in accordance with the present invention.

FIG. 29 is an exemplary view of a light emitting diode (LED) and camera lens arrangement.

FIG. 30 is an exemplary LED assembly.

FIG. 31 is an exemplary graph showing tissue adsorption of methylene blue as a function of wavelength.

FIG. 32 is an exemplary graph showing tissue adsorption of methylene blue as a function of wavelength encompassing five bands/wavelengths.

FIG. 33 illustrates one example of a central processing unit for implementing a computer process in accordance with a computer implemented embodiment of the present invention.

FIG. 34 illustrates one example of a block diagram of internal hardware of the central processing unit of FIG. 33.

FIG. 35 is an illustrative computer-readable medium upon which computer instructions can be embodied.

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

In one aspect of the invention, methods for in situ diagnosis of a disease state in biological tissue or cells of a living organism are provided. The methods take advantage of the discovery herein that the extent of metachromasia determined in biological tissue and/or cells of a living organism stained with a metachromatic dye can be correlated to a disease state. Methods for creating a library of spectra of biological tissue or cells are also provided and may be utilized in the aforementioned methods. Methods for in vivo monitoring, and/or increasing the efficiency, of photodynamic therapy of biological tissue or cells of a living organism are also provided. A bio-spectral imaging system is also provided. Although the imaging system has a wide variety of uses, it may be advantageously utilized in the methods provided herein.

In one form of the invention, a method for in situ diagnosis of a disease state in biological tissue or cells of a living organism includes (a) applying to the tissue or cells in situ a biological stain composition including at least one metachromatic dye; (b) performing a spectral analysis of the stained test tissue or cells to obtain at least one spectrum, preferably obtaining at least one reflected light spectrum of the stained tissue or cells and further preferably determining the degree of the metachromatic shift of the dye from the spectrum; (c) comparing the spectrum from the stained test tissue or cells with the spectra from a library of spectra of similarly stained reference tissue or cells, and preferably comparing the degree of the metachromatic shift of the dye from the spectrum of the stained test tissue and/or cells with the degree of the metachromatic shift of the dye from the library of previously obtained spectra of similarly stained reference tissue or cells, wherein the reference tissue or cells have been previously categorized as diseased or non-diseased; and (d) correlating the reflected light spectrum with a disease state, whereby an in situ diagnosis of a disease state is made.

A wide variety of disease states may be diagnosed according to the above-referenced method. Exemplary disease states that may be diagnosed according to the methods described herein include pre-cancer, including dysplasia; cancer, and specifically including, for example, the pre-cancerous conditions Barrett's esophagus, actinic keratosis and hyperkeratotic conditions generally. Other disease states that may be diagnosed according to the methods described herein include those caused by various microorganisms. In reference to disease states caused by various microorganisms, “diagnosis of a disease state”0 refers to determining the presence or absence of such microorganisms that cause various disease states, including bacteria, fungi and viruses. As mentioned above, in this form of the invention, a diagnosis is made that a bacterial, fungal or viral infection is present. Diseases states caused by bacteria, fungi and viruses are well known to the skilled artisan, and are described, for example, in standard works including, but not limited to, Huntington's Principles of Internal Medicine, which is periodically updated.

A wide variety of such bacteria, viruses and fungi may be detected, including, for example, Gram-positive and Gram-negative bacteria, DNA and/or RNA viruses, and various fungi known to the art. Tissue infected with such microorganisms and stained according to the methods of the present invention may be diagnosed based on the degree of metachromasia, as well as the staining pattern observed within a tissue section.

In a preferred form of the invention, the methods described herein may be used to diagnose pre-cancer or cancer. As known in the art, the term cancer refers to a disease that affects a wide variety of cell types. The disease is characterized by an abnormal proliferation of cells and/or tissues, which may take the form of a tumor. The properties of cancerous cells are well known to the skilled artisan and include exhibition of loss of contact inhibition, invasiveness and the ability to metastasize.

As mentioned above, a biological stain composition that is applied to the desired tissue and/or cells may include a single dye or more than one dye. In a preferred form of the invention, the stain composition includes at least one metachromatic dye. As known in the art, a metachromatic dye is one that exhibits metachromasia (i.e., the situation in which a dye applied to cells or tissues exhibits a color different from that of the dye solution). The extent of metachromasia is dependent on a variety of factors, including variation in mucin production, aberration of nucleic and nucleoli, mitochondrial organelle distribution, charge structure and membrane transport properties. Additionally, it has been discovered herein that the extent of metachromasia exhibited by diseased tissue or cells may be used to diagnose the particular disease state.

A wide variety of metachromatic dyes may be utilized in the invention. Exemplary metachromatic dyes include thiazine dyes. In a preferred form of the invention, the metachromatic dye is a thiazine dye, including methylene blue, toluidine blue O and derivatives or homologues thereof, such as Azure A, Azure B, Azure C and Thionin. Other metachromatic dyes may be selected by the skilled artisan.

Although not intending to be limited by theory, the metachromatic characteristics of thiazine dyes may result from an equilibrium of monomeric, dimeric and/or oligomeric forms of the dye, with the equilibrium being influenced by the local solvent, co-solutes and pH. For example, methylene blue in aqueous solutions is in equilibrium between the monomer form and the dimer form, the ratio between the monomer and dimer being dependent on concentration of the dye and any co-solutes, such as salt, that may be present. Such a ratio can be determined spectroscopically, for example, by using the bio-spectral imaging system of the present invention, or other commercially available imaging system as the peak absorption of the monomer form is centered at about 665 nm and the peak absorption of the dimer form is centered at about 630 nm. Higher oligomer aggregates, if present, will display shoulders or secondary peaks moving toward the blue end of the visible spectrum. As another example, the peak absorption of the monomer form of toluidine blue O is centered about 640 nm and the peak absorption of the dimer form is centered about 600.

Although further not intending to be limited by any particular theory, the color shifts of the metachromatic dyes described herein may, alternatively, result from electron delocalization of the chromophore of these molecules as influenced by stacking where dimer and higher aggregates occur, as well as by charge. In tissue, additional metachromatic shifts may be caused by electrostatic interactions of the dye with biopolymers, such as proteins, including glycoproteins, and nucleic acids.

The dye, or combination of dyes, selected, will depend on the nature of the disease state and the nature of the cells and/or tissue to be stained. For example, in reference to cancer disease states, toluidine blue O is presently preferred for the diagnosis of squamous cell carcinoma of the oral cavity since hemoglobin, which has an absorption at a wavelength that may interfere with the absorption of the metachromatic shift of toluidine blue O, is generally not in the mucosal membranes to which the stain would be applied. As a further example, methylene blue is preferred in diagnosing bladder cancer, Barrett's esophagus, parathyroid adenomas, and pancreatic islet cell tumors.

Other metachromatic dyes, combinations of such dyes, or combinations of metachromatic and non-metachromatic dyes may also be used according to the methods described herein. For example, methylene blue and a pyronin dye, such as pyronin Y, applied either together in a single preparation or in two separate preparations, may afford a desirable degree of enhanced specificity in certain applications. For example, the two stains will compete for certain histochemical features that would distinguish cells undergoing normal repair processes and those cells that are neoplastic, including those that are metaplastic.

Additionally, an eurhodin dye, such as neutral red, may optionally be applied in combination with methylene blue. Neutral red, like methylene blue and toluidine blue 0, has a similar affinity for cancer cells. A combination of neutral red and methylene blue may both stain cancer cells, competing for different cytochemical features of the cells. A spectral analysis of the resulting stain intensity will provide additional information about the disease state of the cells which may be correlated with a histochemical evaluation. This information may be added to the library or database of information and used for subsequent analyses of similarly stained cells.

For example, by comparing the localization of the, for example, eurhodin dye, such as neutral red, with the metachromasia from methylene blue, the analysis may be mutually confirmatory for pre-cancerous and cancerous cells. This would eliminate any possible false positive staining by non-specific dye uptake. If both neutral red and methylene blue can be found within a given tissue region, together with significant metachromasia as provided herein, the evaluation may be more rigorous in selected situations.

In the case of disease states caused by various microogranisms, including bacteria, fungi and viruses, either toluidine blue O or methylene blue are preferred to determine the presence or absence of the microorganisms.

The properties of the stain composition are preferably controlled with respect to concentration, pH, stain ratio and other solvent characteristics. Although the properties can be adjusted depending on the nature of the dye and the samples to be stained as described below, the stain composition typically includes about 0.01% weight/volume (w/v) to about 1% w/v of a metachromatic dye, further preferably about 0.05% to about 0.1% w/v. If other dyes are present in the compositions, they are typically present in the compositions at a weight ratio of the other dye to the metachromatic dye of about 1:5 to about 1:1, preferably about 1:2 to about 1:3. The pH of the composition will depend on the nature of the dye utilized, but may typically range from about 4 to about 5, preferably about 5.5 and may be maintained by buffer compositions known to the skilled artisan, including acetate buffer. In the case of thiazine dyes, for example, the pH is preferably less than 7, as thiazine dyes are less stable at an alkaline pH than at an acidic pH.

The preferred stain concentration may vary depending on the nature of the stain, the amount of time the tissue or cells are stained and the destaining technique. For example, in order to stain oral mucosa to diagnose oral cancer, the stain composition preferably includes toluidine blue O at a concentration of about 1% w/v in acetate and a pH of about 6. As a further example, when determining the margins of a basal cell carcinoma ex vivo, the freshly excised tissue is preferably stained with an about 0.05% w/v solution of methylene blue. It is preferred to prepare the methylene blue composition as a hydrochloride salt, so that pH is slightly acidic.

A wide variety of tissues and/or cells may be diagnosed according to the methods of the present invention. Exemplary tissue and/or cells may be derived from, for example, skin, cervix, vagina, oral cavity, colon, esophagus, and bladder, as well as other desired internal organs. Preferably, the tissue and/or cell samples are derived from epithelium. The cell samples utilized, including those that form the tissue utilized in the methods described herein, are typically whole, or otherwise intact, cells. For example, the cells preferably have an intact cell membrane. Therefore, it is preferred that the cells are not lysed or otherwise treated to disrupt their membranes.

The tissue and/or cell samples are typically analyzed in situ. As known in the art and as described herein, in situ analysis refers to in vivo or ex vivo (freshly excised) analysis. By “freshly excised” is meant that the samples are spectroscopically and/or histochemically analyzed no more than about 20 minutes, preferably no more than about 5 minutes, after being removed from the patient. The freshly excised samples are typically analyzed about 1 minute to about 5 minutes after being removed from the patient.

Prior to staining the tissues and/or cells with the dye composition, the area to be stained may be cleaned by cleaning procedures known to the skilled artisan, such as by cleaning with an alcohol preparation on applicators, such as a cotton swab. Additionally, prior to staining the specific area, one or more spectra, such as reflectance spectra, are preferably obtained of the area to be stained to determine a background spectrum over the range of wavelengths that is optimal for the specific application. Moreover, prior to staining the tissue and/or cell samples, it is preferred that the samples remain unfixed, unlike traditional staining methods. Such an unfixed state is preferred as transverse thin sections, which are often employed in conventional histopathological diagnosis using conventional staining techniques, are orthogonal to the cell membranes, leaving little of the membrane, other than the edge, presented for viewing. A fixed thin section does not have an active cell membrane potential to exclude or include the dye in an active manner, nor does it have a sufficient cell membrane presented to the viewer to determine the extent of dye localization within the membrane.

For example, the sensitivity of thiazine dyes for pre-cancerous and cancerous cells is determined by the properties of the dyes, such as lipophilicity, cationicity, and molecular weight. The lower the molecular weight of a dye, the more readily the dye is taken up by the cell membrane of diseased cancer cells. Pre-cancerous and cancerous cells have a permeability for thiazine dyes in excess of the chemical activity needed for transport across a gradient potential. While not intending to be bound by any particular theory, it is believed that the metachromasia associated with this effect may be 1) a concentration effect, in which higher inner membrane concentration of the thiazine dyes causes the shift from monomeric to oligomeric forms, producing metachromasia, 2) that the association of the dyes with cell membrane components, such as the highly charged phospholipids, causes electron delocalization and a shift in the absorption peak of the thiazine chromophore, or 3) a combination of these effects.

After the optional pre-cleaning procedure, the stain composition may then be applied by an applicator, such as cotton or other similar applicator known to the skilled artisan, and allowed to remain on the area for a time period sufficient to stain the cells so that any metachromasia may be observed. Although this time period may vary depending on the concentration of the dye, the nature of the dye, and the tissue being stained as known in the art, the stain typically remains on the area from about 0.5 minute to about 5 minutes, and further preferably from about 0.5 minutes to about 1 minute.

Excess stain is then preferably removed from the areas which do not biochemically, cytochemically or otherwise retain the stain by de-staining procedures known to the art, including use of a cotton or other applicators known to the art. The length of the destaining procedure is sufficient to remove all excess dye as determined visually. Although this time period may vary depending on the nature of they dye, the concentration of the dye applied, the extent of the area stained and the nature of the tissue stained, destaining is typically performed for a period from about 0.5 minutes to about 5 minutes, preferably from about 0.5 minutes to about 1 minute.

As one example of the staining/destaining procedure for in situ determinations of actinic keratosis or squamous cell carcinoma, an aqueous solution of about 0.05% w/v methylene blue may be may be applied directly to the suspected lesion with a methylene blue-saturated cotton applicator and allowed to remain on the lesion for about 0.5 minutes to about 1 minute. Excess stain may be removed with a cotton or other applicator with a solution of 2% acetic acid and 5% ethanol in water. Other suitable destaining solutions are known to the skilled artisan.

Once the staining and optional destaining have been performed, a spectral analysis of the stained tissue or cells using, for example, the bio-spectral imaging system shown in FIGS. 28-30, is performed to obtain at least one spectrum. In preferred forms of the invention, the metachromasia brought about by, for example, interaction of the dye with the cell and its cellular constituents is measured by a spectroscopic method, such as by reflectance spectroscopy. When performing the diagnosis in vivo on the skin, for example, the skin may be illuminated with the desired light source and the spectrum may be generated as known in the art. When performing the diagnosis inside the body, it may be desirable to utilize appropriate catheters or other tubing in order to stain/destain the tissues and illuminate the tissue to subsequently generate the desired spectrum. As a more specific example, intra-arterial injection may be utilized, for example, for pancreatic islet cell tumors. Intravesicular instillation may be utilized for bladder tumors. In the case of conditions affecting the esophagus, the stain may be sprayed onto the esophagus. Additionally, intravenous injection may be utilized to observe abnormal parathyroid glands.

Once the spectrum, such as the reflected light spectrum, of the test samples is obtained using, for example, the bio-spectral imaging system shown in FIGS. 28-30, various spectral features of the spectrum may be recorded or otherwise noted. For example, the degree of the metachromatic shift of the dye from the spectrum may be obtained. In preferred forms of the invention, this may be obtained by comparing the intensity of the reflected light between, for example, at least two wavelengths of the light spectrum. As one example, with respect to methylene blue, one can compare the intensity at 610 nm relative to 640 nm and 670 nm. Other examples of specific spectral features that may be observed are discussed in greater depth in, for example, Example 1.

The spectral features from the spectrum, such as the reflected light spectrum, of the test sample are compared to the spectral features obtained for one or more similarly stained reference samples. For example, the extent or degree of the metachromatic shift of the dye from the spectrum of the test sample may then be compared with the extent or degree of the metachromatic shift of spectra of one or more reference samples that are preferably taken from the same type of tissue or organ being studied. As a more specific example, if a diagnosis of skin cancer is being made, the reference sample is preferably also from the skin. It will be appreciated that the one or more reference samples may be obtained from the same individual or organism as is the test sample. Thus, for example, a baseline or “normal” reflected light spectrum for a given individual may be obtained by taking one or more samples, either at the same time or preferably over a period of time, and these may comprise the reference against which the reflected light spectrum for test samples taken from the same individual at a later point in time may be compared. More typically, however, the reference samples will be derived from groups of individuals, and the reflected light spectrum data from these individuals will be pooled such that a database or library may be created. The similarly stained samples that this library of previously obtained spectra are based upon will have been previously categorized as diseased or non-diseased, and preferably will have been categorized as to the extent of the disease (e.g., with respect to cancer, the cells may be pre-cancerous or cancerous). By “non-diseased” is meant that the specific disease state has not been found with respect to the sample. For example, when diagnosing a disease such as cancer, the term non-diseased or normal as used herein indicates that the tissue and/or cell sample is not pre-cancerous or cancerous.

The samples upon which the library is built may be categorized, or otherwise diagnosed, as diseased or non-diseased by a variety of methods. It is presently preferred that a pathologist utilize conventional procedures to make such a determination. In certain forms of the invention, the diagnosis may be made by conventional histological techniques, including conventional histochemical and/or biochemical techniques. For example, the sample may be sectioned if necessary, frozen (such as in a cryostat) and stained utilizing biological dyes or stains known to the art for such purpose. Exemplary biological stains include toluidine blue O and haematoxylin-eosin. Other suitable stains are known to the skilled artisan. After staining, a diagnosis is made, for example, by a pathologist by examination of the stained sample under a microscope, such as a high power microscope or other similar device, to determine the presence of, absence of, or change in various cellular and/or sub-cellular structures.

As mentioned above, various spectral features in the library or database of spectra, such as reflected light spectra, are recorded or otherwise noted. In preferred forms of the invention, the spectral features of interest include the intensity of the reflected light between, for example, at least two wavelengths. As one example as mentioned above, with respect to methylene blue, one can compare the intensity at 610 nm relative to 640 nm and 670 nm. In certain forms of the invention utilizing methylene blue, the wavelengths, the intensity of which is examined, may preferably be chosen towards the blue end of the spectrum from, for example, about 610 nm to about 640 nm. A series of what is referred to herein as “metachromatic indices” may then be generated, as more particularly described in Example 1, and may act as the basis for comparison of the samples.

For example, with respect to methylene blue and diagnosing precancer or cancer, a metachromatic index (MI) of 0 leads to a diagnosis of non-diseased, and refers to the fact that there is no indication of peak broadening to wavelengths shorter than the center wavelength of 670 nm. If the intensity at 610 nm is equal to or exceeds that at 640 and 670 nm, (MI class I) an indication of cancer is present. As mentioned above, the spectral features that define such indices are more fully described in Example 1. Once the spectral features from the spectrum of the test sample are determined and compared to the spectral features of the spectrum of the reference samples, a correlation as to disease state may be readily made.

Although not intending to be bound by any particular theory, it is believed that what is being quantitated from the spectra are the relative amounts or proportions of monomeric, dimeric, and higher oligomeric aggregates of the dye present in the dye-stained samples as more fully described herein. Determining the degree of the metachromatic shift of the dye from, for example, the reflected light spectrum of the test tissue and/or cells, as well as comparison of this degree of the metachromatic shift with the degree of the metachromatic shift of the reflected light spectrum of the reference samples, may be accomplished with the use of computer software known to the art, including Multi-Spec and Envi. Accordingly, the database, or library, may include a digital spectrum library, and/or a library of desired spectral features as described herein, stored in a computer. In other forms of the invention, the spectroscopic data may be compared graphically or by other similar methods. If desired, a background adjustment may be made by having the software subtract from the spectra analyzed the background reflectance spectra of normal tissue, both with and without stain, including a baseline spectrum of the patient's normal tissue.

In yet another aspect of the invention, methods of creating a library of spectra of biological tissue or cells of a living organism are provided. In one form, a method includes (a) applying to the tissue or cells a biological stain composition including at least one metachromatic dye to form stained tissue or cells; (b) determining whether said stained tissue or cells are diseased or non-diseased; (c) performing a spectral analysis of the stained tissue or cells to obtain at least one spectrum, such as a reflected light spectrum; (d) analyzing the spectral features of said spectrum; and (e) correlating said spectral features to said disease state. The biological stains as previously described herein may be used. Additionally, in preferred forms of the invention, analyzing the spectral features of the reflected light spectrum preferably includes obtaining the metachromatic shift of the dye of the stained reference tissue. Determining whether the stained tissue or cells are diseased or non-diseased is preferably accomplished by analyzing the samples by conventional histological techniques, preferably by a pathologist, as previously described herein. The spectral features are further preferably correlated to the disease state by matching the spectral features to the disease state and storing this information in a computer database or other library as previously described herein.

In yet other aspects of the invention, methods for monitoring the efficiency of photodynamic therapy of diseased biological tissue or cells in situ are provided. In one form of the invention, a method includes (a) applying to the tissue or cells a phototosensitizing metachromatic dye to form stained test tissue or cells, (b) performing a spectral analysis of the stained test tissue or cells to obtain at least one spectrum, preferably by obtaining a reflected light spectrum of the stained tissue or cells and further preferably determining the degree of the metachromatic shift of the dye from the spectrum; (c) comparing the spectrum, such as the reflected light spectrum, from the stained test tissue or cells with the spectra from the library of spectra of similarly stained reference tissue or cells, preferably by comparing the metachromatic shift of the dye from the spectrum of the stained test tissue or cells with spectra of similarly stained reference tissue or cells that have been previously categorized as diseased or non-diseased; (d) irradiating the stained tissue or cells with light having an intensity sufficient to induce photooxidation of said dye, and (e) monitoring the changes in the metachromatic shift of the dye by repeating steps (b) and (c).

In preferred embodiments of the invention, the initial steps of this method are similar to that previously described for the in situ diagnosis of a disease state in that a stain or other dye is applied to the tissue and/or cell test sample, and a spectral analysis is performed on the test sample to obtain at least one spectrum which is compared to spectra of similarly stained reference tissue and/or cells that have been previously categorized as diseased or non-diseased. Similarly, in further preferred forms of the invention, the spectral analysis includes determining the extent of the metachromatic shift of the dye from, for example, the reflected light spectrum of the test sample. Additionally, the extent of the metachromatic shift of the dye from the spectrum of the test sample is preferably compared to the metachromatic shift of the dye from the spectra of the similarly stained reference tissue or cells that have been previously categorized as diseased or non-diseased. Moreover, in one preferred form of the invention, the method of monitoring the efficiency of photodynamic therapy includes irradiating the stained tissue or cells with light having an intensity sufficient to induce photooxidation of the dye and monitoring the changes in the metachromatic shift of the dye by monitoring and analyzing the spectra, such as the reflected light spectra, as a function of time by repeating steps (b) and (c) described in the preceding paragraph.

The method may be practiced to monitor the efficiency of photodynamic therapy of a wide variety of diseased biological tissue or cells, including biological cells diagnosed as pre-cancerous (including dysplastic) and cancerous, or other disease state known to the art that is amenable to treatment with photodynamic therapy. Additionally, the photosensitizing metachromatic dyes utilized in the monitoring method are identical to those previously described herein, and other such dyes would be known to the skilled artisan.

A wide variety of light sources may be utilized to irradiate the stained tissue and/or cells, including broad-spectrum high intensity light sources, a filtered high intensity light source, specific wavelength lasers, or other light sources known to the skilled artisan. Exemplary light sources include solid state light emitting devices. A preferred light source is a light emitting diode of a wavelength coincident with the peak absorbance of the specific dye. As one example, such a wavelength would be about 665 nm for methylene blue. The power of the irradiating light source applied will depend, for example, on the nature of the disease being treated, the nature and amount of the photosensitizing dye used, the length of the treatment and the nature of the source of irradiation. Typically, the power of the irradiating light is from about 2 mW to about 100 mW, preferably about 5 mW to about 20 mW.

After irradiation, in further preferred forms of the invention, the changes in the metachromatic shift of the dye are monitored. In order to accomplish the monitoring in certain embodiments of the invention, a spectrum, such as a reflected light spectrum, of the stained tissue or cells is obtained, the extent of the metachromatic shift of the dye from the spectrum is determined and is compared with the extent of the metachromatic shift of the dye from the spectra, such as reflected light spectra, of similarly stained tissue or cells that have been previously categorized as diseased or non-diseased, all as previously described herein. The change in the respective spectra, such as the change in the intensity of the reflected light at one or more wavelengths, may then be monitored as a function of time.

For example, in the case of diagnosing dysplasia, pre-cancer or cancer utilizing methylene blue, the relative intensity at 610 nm compared to 640 nm and 670 nm may be monitored. An objective is to decrease and preferably eliminate the metachromatic effect which will indicate that substantially all of the diseased tissue, such as greater than about 95%, preferably greater than about 99% and preferably about 100%, of the diseased tissue has been killed or otherwise removed and/or all of the dye has been photooxidized. For example, with respect to methylene blue, the objective is to have a spectrum of non-diseased tissue and/or cells at the end of the photodynamic therapy session, wherein there, for example, is no indication of peak broadening to wavelengths shorter than 670 nm. Such a method therefore indicates the status, or progress, of the therapy. In other forms of the invention, changes may be made in the phototherapy procedure in order to increase the efficiency of the therapy.

Accordingly, in yet another aspect of the invention, methods of increasing the efficiency of photodynamic therapy of diseased biological tissue or cells in vivo are provided. As in the method of monitoring the efficiency of photodynamic therapy described herein, the method includes (a) applying to the tissue or cells a phototosensitizing metachromatic dye to form stained test tissue or cells, (b) performing a spectral analysis of the stained test tissue or cells to obtain at least one spectrum, preferably by obtaining a reflected light spectrum of the stained tissue or cells and further preferably by determining the extent of the metachromatic shift of the dye from the spectrum of the stained test tissue or cells; (c) comparing the spectrum from the stained test tissue or cells with the spectra from the library of spectra of similarly stained reference tissue or cells, preferably by comparing the metachromatic shift of the dye from the reflected light spectrum of the stained test tissue or cells with reflected light spectra of similarly stained reference tissue or cells that have been previously categorized as diseased or non-diseased; (d) irradiating the stained tissue or cells with light having an intensity sufficient to induce photooxidation of said dye, and (e) monitoring the changes in the metachromatic shift of the dye by repeating steps (b) and (c).

In addition, in one form of the invention, the intensity of the irradiating light or the duration of the irradiation of the stained test tissue or cells may be adjusted, or the source of the irradiating light may be repositioned, based on the difference between the spectra of the test tissue or cells and that of the reference tissue or cells. For example, the power of the laser could be increased if the photooxidation is proceeding slowly such that the rate of change in, for example, the intensity of the 610 nm peak relative to the 640 and/or 670 nm peak is slower than desired. The power of the laser should not exceed that which would otherwise cause injury to non-diseased tissue of the patient. The power of the irradiating light, including the power above which injury to non-diseased tissue may occur, is known in the art and is dependent on the nature of the disease being treated, the nature and amount of the photosensitizing dye used, the length of the treatment and the nature of the source of irradiation.

Typically, the power of the irradiating light is from about 2 mW to about 100 mW, preferably about 5 mW to about 20 mW. Moreover, the duration of the photodynamic therapy is also dependent on the situation, but may be easily determined by the skilled artisan. Additionally, analysis of the spectra will reveal when all of the dye has been photooxidized, and thus when the treatment should be stopped. This may decrease the time the patient is unnecessarily exposed to the light source, thereby decreasing the chance of damaging non-diseased tissue.

Furthermore, monitoring the course of the photodynamic therapy allows one to distinguish between diseased and adjacent, non-diseased tissue. Therefore, one can reposition the light source so that only diseased areas will be irradiated. For example, in Mohs surgery, one can determine the margins of a particular skin carcinoma, and consequently, when all of the diseased tissue has been killed or otherwise removed.

In yet another aspect of the invention, a bio-spectral imaging system is provided. The bio-spectral imaging system have a variety of uses. In one preferred form of the invention, the bio-spectral imaging system may be utilized in the methods described herein. Referring now to FIG. 28, an exemplary elevation view of an embodiment of the metaspectral imaging system 28 is shown, in conjunction with a general purpose computer system 104. As previously discussed, the system 28 can be utilized for, inter alia, recording the spatial distribution of metachromatic staining of freshly excised surgical tissue on a microscope slide when, for example, toluidine blue O or methylene blue is used as a stain.

The system 28 optionally utilizes a base 14 upon which, for example, a rack 10 can be attached to facilitate vertical movement of housing 19 and/or camera holder 15. The housing 19 contains a light emitting diode (LED) assembly 9, and camera holder 15 holds, for example, camera 8, which is preferably a digital camera. A pinion (not shown) associated with each of the LED assembly mount 13 and camera mount 18 can be utilized in conjunction with rack 10 to respectively move the housing 9 and/or camera holder 15. Other securing means, such as a friction holding, screw holding, and the like, can also be used.

The housing 19 can be secured to LED assembly mount 13 by a securing means 17 such as a pin, dowel, screw, or bolt. The housing 19 can also be adhesively secured to LED assembly mount 13. The camera holder 15 can be similarly secured to camera mount 18.

Camera 8, which has a lens 1 with first 1a and second 1b opposing end positions, is positioned within camera holder 15. In at least one embodiment, the camera 8 is used to measure light reflected from the sample on slide 4 when the sample is illuminated, preferably by at least two LEDs 6 having a substantially same wavelength in the range of approximately 200 nanometers (nm) to approximately 1100 nm. The use of a single LED 6 can also be used to illuminate the sample on slide 4.

In at least one embodiment, the camera lens 1 is a close focus lens, preferably having a magnification of approximately 1 or higher. The lens 1 can be, for example, a 60 mm F2 Nikkor lens, and the camera 8 can be, for example, a Nikon D1X digital camera. Other camera 8 and lens 1 combinations with other characteristics can also be utilized.

The diameter of lens 1 is smaller than the diameter of light baffle 2, which preferably contacts a bottom portion of the LED assembly 9. The lens 1 is free to move along the optical axis substantially orthogonal to the image plane 3.

A light trap 12 is preferably used to cover the sample on the slide 4. The light trap 12 is preferably has a flat black inside surface that is exposed to light from the LEDs 6 to prevent internal reflection of the light passing through or around the sample on slide 4. The light trap 12 thus provides a dark field background, and provides contrast to the sample on slide 4. The light trap 12 also substantially eliminates trans-reflectance through the tissue sample on slide 4.

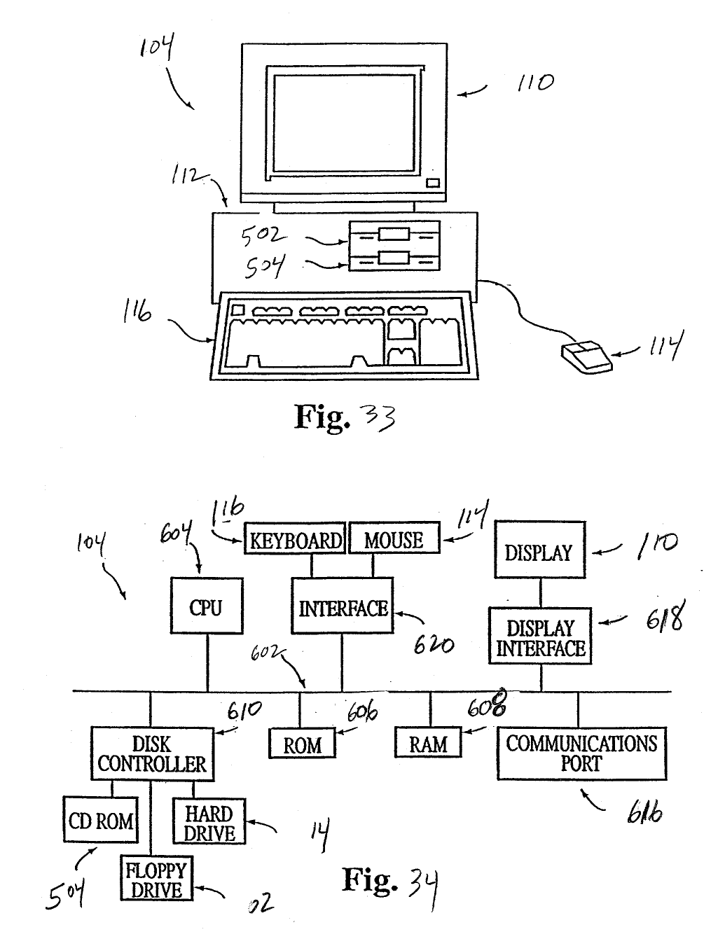

FIG. 28 also shows a computer system 104, which receives data from the camera 8 by using, for example, a connection 24 such as a standard Institute of Electrical and Electronics Engineers (IEEE) 1394 connection. The computer system 104 will be further discussed with regard to FIGS. 33 and 34.

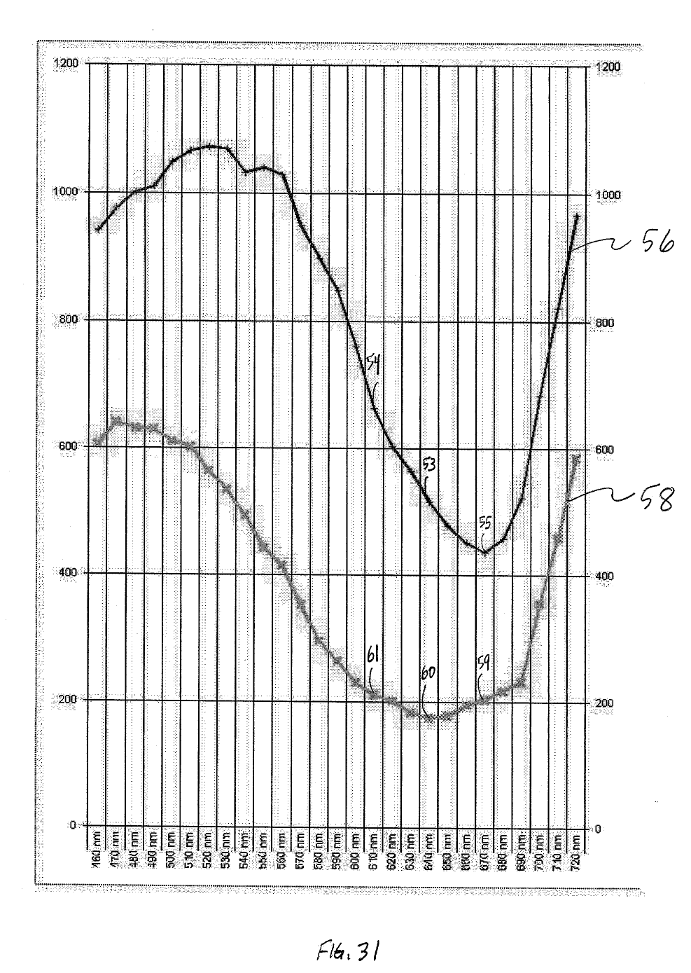

FIG. 29 shows an exemplary embodiment of the light emitting diode assembly 9 in relationship to the lens 1 and light baffle 2. As shown, the lens 1 is positioned and sized to enable movement within and relative to the light baffle 2. The LED assembly mount 13 and/or camera mount 18 can be moved to position the end of lens 1a a designated distance from slide 3.

In at least one embodiment, the slide 4 and LED assembly 9 together move as the LED assembly mount 13 moves. The LED assembly 9 has a plurality of LEDs 6, each optionally having a holographic diffuser 7. Uniformity of beam illumination from each LED 6 can be improved by using a holographic diffuser 7 or other suitable light shaping plate(s). LEDs 6 are available from, for example, Agilent Technologies, Palo Alto, Calif., Lumex Corporation, Palatine, Ill., and/or Fairchild Semiconductor, South Portland, Me. Holographic diffusers 7 are available from, for example, Physical Optics Corporation, Torrance, Calif.. The LEDs 6 are preferably mounted on an angled surface 20 to focus the light from each activated LED 6 on the slide 4. As shown in FIGS. 29 and 30, surface 22 is integral with angled surface 20, and extends vertically relative to flat portion 23. The wall of the unit 25 is preferably positioned between the LEDs 6 and the sample stage 51, and is preferably integral to the angled surface 20 in which the LEDs 6 are mounted.

FIG. 30 is an exemplary top view of an LED assembly 9. In FIG. 30, the LED assembly 9 has twenty four (24) LEDs 6 mounted on the angled surface 20. Other quantities of LEDs 6 can also be utilized. An aperture 5 is preferably formed within flat portion 23 of the LED assembly 9. The angled surface 20 is preferably at an angle of approximately 45 degrees with respect to the horizontal so that the axis of the light beams emanating from activated LEDs 6 is directed to the sample on slide 4. It is preferred that substantially all light from the LEDs 6 converge over the FOV (e.g., 11.5 mm×9.2 mm) at the image plane 3. The LEDs 6 can optionally be mounted in a portion of a printed circuit board 11. The circuit board 11 can have, for example, a conventional RS-232 connection (not shown) that interfaces to computer system 104, enabling software running on computer system 104 to control, for example, the timing and/or sequencing of LEDs 6. Software running on computer system 104 can also control operation of camera 8 such that the camera 8 takes a picture of the sample on slide 4 when the sample is illuminated by one or more of the LEDs 6 having a particular wavelength. Timing and sequencing information can be entered by a user using, for example, the keyboard 116 and/or mouse 114 of computer system 104. The computer system 104 receives data from the camera 8 and transmits control commands to the computer system 104 by using, for example, a connection 24 such as an IEEE 1394 connection. The computer system 104 will be further discussed with regard to FIGS. 33 and 34.

In at least one embodiment, at least two LEDs 6 at a given wavelength provide substantially uniform illumination of the sample on slide 4. To achieve uniform illumination, it is preferred that the beam width (selected from the LED specifications) of each of the at least two LEDs 6 are the same. The holographic diffuser 7 optionally utilized in conjunction with an LED 6 makes substantially uniform illumination across the image plane 3.

With regard to FIG. 30, illustrating an exemplary embodiment having twenty-four (24) LEDs 6 mounted on the angled surface 20, there can be, for example, twelve matched sets of two LEDs 6, where each of the sets of two LEDs 6 preferably have substantially the same wavelength and beam width. Providing at least two LEDs 6 at a selected wavelength advantageously provides a more uniform illumination of the sample on slide 4, and also provides redundancy in the event one (or more) LED(s) 6 at a particular wavelength is rendered inoperable. In at least one embodiment, each of the two LEDs comprising each set are spaced approximately 180 degrees apart on angled surface 20. Similarly, there can be, for example, six matched sets of four LEDs 6, where each of the sets of four LEDs 6 preferably have substantially the same wavelength and beam width. Each of the four LEDs comprising each of the six sets would be spaced approximately 90 degrees apart on angled surface 20. For a given type of stain, increasing the number of matched sets of LEDs 6 having different wavelengths enables examination of the spectral characteristics of the sampled tissue at a greater number of wavelengths, and accordingly increases the statistical confidence of identifying, for example, normal and abnormal tissues, and/or variants thereof.

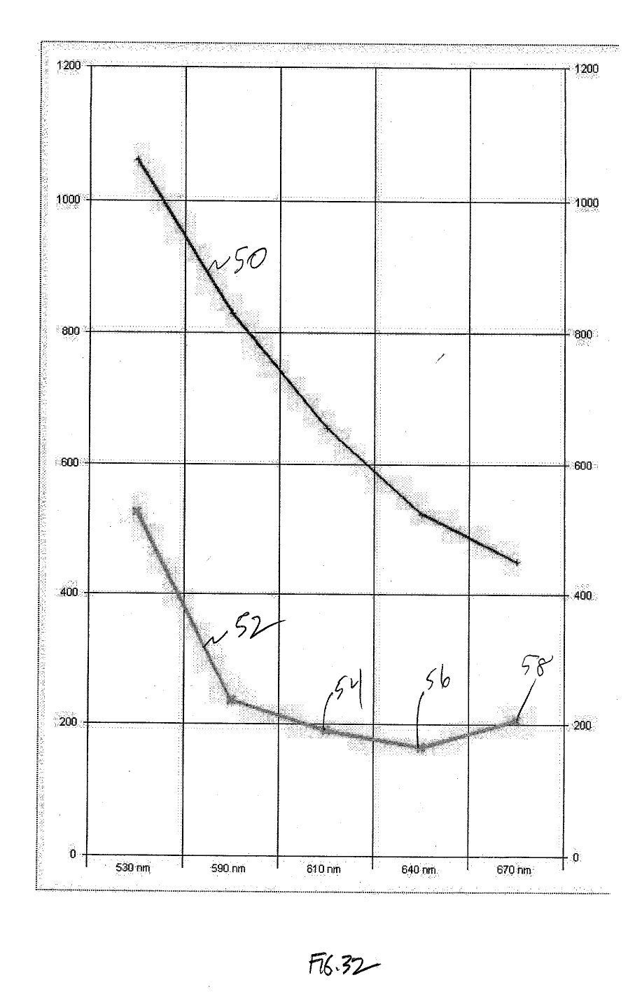

FIG. 31 is an exemplary chart showing the light reflected from a portion of a tissue sample on slide 4, as a function of wavelength. The portion can correspond, for example, to a single pixel or group of pixels comprising a region of interest (ROI) of the charge coupled device (CCD) (not shown) of camera 8. In particular, the abnormal tissue utilized to obtain the data points of curve 58 shown in FIG. 31 was a skin lesion, having been previously diagnosed as a basal cell carcinoma. Subsequent to excision, the tissue was rinsed in 0.1 M acetate buffer pH 4.5 for 30 seconds, and then rinsed for 30 seconds in 0.05% aqueous methylene blue. Excess methylene blue was removed by a 30 to 60 rinse in 1% acetic acid. A similar skin lesion was used to obtain the data points of curve 56. Other preparation techniques and/or processes can also be utilized.

The y-axis of FIG. 31 is the binary or raw response of each pixel (or an average raw response of a ROI) of the CCD (not shown) of camera 8. For example, suppose the CCD (not shown) of camera 8 has a 12 bit response and therefore records the intensity of light as an integer between 0 and 4096. In this case, if the pixel (or ROI) is saturated with light, the reading is 4096. If no light is received, the reading is 0. The x-axis of FIG. 31 shows the wavelength of one or more of the LEDs 6 that illuminate the sample on slide 4.

The system 28 can be used to record the data for the sample (or portion thereof) on slide 4. In at least one embodiment, for any given pixel (or ROI), the results are indicative of only a normal portion 56 or only an abnormal portion 58.

As shown in FIG. 31, data was recorded using LEDs 6 having wavelengths from 460 nm to 720 nm, in 10 nm increments. In operation, it is preferred that the camera 8 record the respective image/data for each respective sample, at each wavelength, and store the data on a storage medium readable by computer system 104. The connection 24 can be, for example, an IEEE 1394 connection used to transmit the data to the computer system 104. It is preferred that the data from the sample captured at each wavelength be transferred to computer system 104 prior to capturing data at a next wavelength.

As shown in FIG. 31 at data point 55, methylene blue has a peak adsorption at 670 nm for the pixel (or ROI) of the normal tissue 56. In general, methylene blue has a maximal light absorption at 665-670 nm. As is also shown in FIG. 31, at data point 60, methylene blue has a peak adsorption of 640 nm for the abnormal tissue 58.

In accordance with the present invention, the relative ratio of light adsorption by the tissue sample at selectively chosen wavelengths can be used to differentiate normal tissue 56 from abnormal tissue 58 portions of the tissue sample. In particular, we have discovered that the peak adsorption point 55, and corresponding peak adsorption point 60 associated with the abnormal tissue 58 can be utilized in conjunction with other data points in determining respective normal and/or abnormal portions of a tissue sample and/or group of cells.

With regard to FIG. 31, the intensity of the normal tissue 56 at 670 nm, 640, nm and 610 nm is approximately 435 (55), 515 (53) and 640 (54), respectively. Using the peak adsorption point 55 as the denominator, the two ratios associated with these three data points are 1.18 (515/435) and 1.47 (640/435), respectively.

With regard to the abnormal tissue 58, the intensity at 670 nm, 640, nm and 610 nm is approximately 200 (59), 180 (60) and 210 (61). Again using the intensity associated with wavelength 670 nm as the denominator, the two ratios associated with these three data points are 0.9 (180/200) and 1.05 (210/200), respectively.

It has been discovered that for a normal tissue 56, as wavelength decreases approximately 60 nm from the wavelength at which maximum adsorption occurs, there is generally an increasing ratio of intensity. Similarly, it has been discovered that for an abnormal tissue 58, as wavelength decreases approximately 60 nm from the wavelength at which maximum adsorption occurs (in normal tissue), there is a ratio of intensity that is approximately 1.0.

Using intensity ratios and two or more data points as discussed above, various algorithms can be used in characterizing the tissue as being normal or abnormal. In addition, the methods in accordance with the present invention are not limited to a binary characterization of tissue as being characterized as normal or abnormal. For example, a third category of “suspected abnormal” can be provided, as can a fourth category of “suspected normal.” These categories would represent that is likely, but not highly likely or conclusive, that the portions of the tissue sample are abnormal and normal, respectively. Other categories can be used in lieu of or in addition to those discussed.

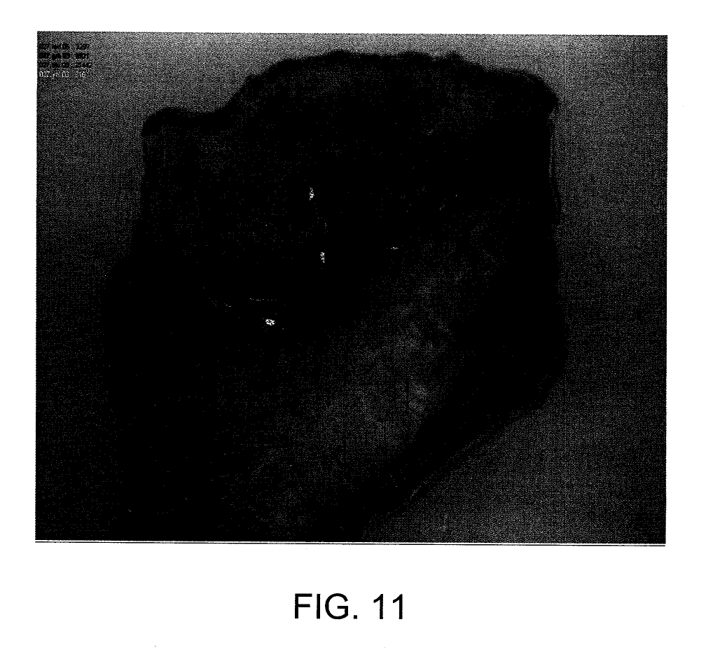

FIG. 32 shows the data points of FIG. 31 at wavelengths of 530, 590, 610, 640 and 670 nm. Pixels (or an ROI) characterized as abnormal 52 can be displayed, for example, on display monitor 110 as relatively bright colored pixels (e.g., red), overlaying a grayscale image of the entire tissue sample, or a portion thereof. As shown in FIG. 11, area 72 (indicated as a red area on FIG. 11) can, for example, indicate abnormal cells, areas 74 (indicated as blue areas on FIG. 11) can, for example, indicate suspected abnormal cells, and areas 76 (indicated as green areas on FIG. 11) can, for example, indicate suspected normal cells. Any color scheme can be used to represent various cell conditions. For example, abnormal tissue 52 can be displayed, for example, on display monitor 110 as blue pixels, instead of red.

As previously discussed, an analysis is done, preferably by software running on computer system 104 and in accordance with predetermined algorithms, on each pixel (or ROI) at each respective wavelength that compares the relative ratio of light adsorption by the stained tissue sample at, for example, each of the three bands. In this regard, it should be understood that there may be hundreds, thousands or millions of data sets such as shown in FIGS. 31 and/or 32 corresponding to a particular sample on slide 4. The adsorption intensity of the dye-stained lesion can then be determined at the principle wavelengths corresponding to the key spectral features of the metachromatic properties of the dye. Using a configuration described above (e.g., a Nikon D1X Digital camera with an array size of 23.7 mm by 15.6 mm having 5.47 megapixel CCD, and a 60 mm F2.8 micro Nikkor lens), a computer system 104 having a CPU 604 with, for example, a 700 MHz clock speed and 512 MB of RAM 608, an image such as shown in FIG. 11 can be displayed in approximately 90 seconds.

In general, for any spectral analysis, additional wavelengths may be measured if blood, for example, is found on a tissue sample. By measuring the adsorption of the blood hemoglobin, any interference of the hemoglobin to the metachromatic analysis of methylene blue or toluidine blue O staining can be determined, and a correction to the analysis made. Hemoglobin shows a strong adsorption in the range of 525 nm to 570 nm and thus a relative intensity of adsorption in this spectral range would assist in providing a correction for adsorption at longer wavelengths.

In operation, the system 28 is used to begin the imaging process preferably by verifying that the slide 4 is within the FOV of the camera 8. Imaging software running on computer system 104 can be used to determine whether a sample on slide 4 is within the FOV. Once the system 28 determines that the slide is within the FOV, information pertaining to the sample is entered into the computer. The information can include, for example, the name of the patient, sample type (e.g., skin), patient identification number, and/or physician name, etc.

The camera 8, optionally under the control of software running on computer system 104, then takes a series of preferably at least three pictures, preferably at the characteristic spectral bands for a particular stain. For example, when methylene blue E is used, LEDs 6 having wavelengths of approximately 670 nm, 630 nm and/or 610 nm can be utilized. It is preferred that the images be stored using the Digital Imaging and Communications in Medicine (DICOM) standard, which was created by the National Electrical Manufacturers Association (NEMA) to aid the distribution and viewing of medical images such as Computer Tomography (CT) scans, Magnetic Resonance Imaging (MRI), and ultrasound. A single DICOM file, corresponding to each picture taken at a given wavelength (e.g., 670 nm) contains both a header (which stores information about the patient's name, the type of scan, image dimensions, etc.), as well as all of the image data (which can contain information in three dimensions). It is preferred that each picture taken by camera 8 be downloaded to the computer system 104 before another picture is taken. An IEEE 1394 connection can be used to transfer data between the camera 8 and the computer system 104 (and vice versa) for eventual display on display 110 of an RGB composite image can be displayed on display 110 corresponding to the raw data taken at a particular wavelength (e.g., 670 nm).

For example, typical results of methylene blue stained, imaged and analyzed basal cell carcinoma tissue section is shown in FIG. 11. In this case, area 72 shows the location of cancerous tissue. The positive results indicating the presence of cancer on or near the margin of the tissue directs a surgeon in further treatment of the tumor. Areas 74 can indicated suspected cancer, and area 76 can indicate suspected normal tissue. Areas 72, 74, 76 can be determined by using the techniques described with regard to FIGS. 31 and 32.

In addition to skin samples, the system 28 can be used to analyze, for example, mucosal smears. For example, epithelial cells from mucosal membranes are often sampled, fixed, and stained on a microscope slide 4 for visual reading to determine if any of the sample cells are dysplastic, pre-cancerous, or cancerous. Both cervical and oral mucosal smears can be analyzed. Methylene blue and toluidine blue O are also known to differentially stain normal and cancerous epithelial cells. By staining a suspension of mucosal sampled epithelial cells with methylene blue or toluidine blue O and collecting the cells on a microporous membrane filter, the differentially stained cells can then be imaged using the system 28, using an appropriate subset of spectral wavelengths. This advantageously enables mucosal and/or epithelial cells to be quickly screened almost immediately after sampling.

The methods in accordance with the present invention can also be adopted to endoscopic methods. Fiber optic imaging systems, such as used in colposcopy, can be adapted with an illumination light guide that is attached to a light emitting diode array having selected LEDs, whereafter image acquisition and analysis using, for example, intensity ratios can be performed as previously described.

In addition, the methods in accordance with the present invention can also be adapted to in vivo imaging. For example, a system such as shown in FIG. 28 can be constructed that facilitates imaging tissue directly on a patient. With an image plane 3 and optics (e.g., LEDs 6) appropriately adjusted, the interaction of methylene blue or toluidine blue O with human tissue directly on the patient would advantageously facilitate, for example, the determination of a surgical margin being free of tumor cells. A relatively large set of LEDs 6 to provide for greater intensity of illumination at each specific wavelength may be utilized together with reducing the ambient background light during imaging. More particularly, video endoscopes having a digital imager may be adapted for in vivo applications such as a digitally imaging colposcope (preferably used for imaging the cervix) or oral camera (preferably used for imaging the oral cavity). For such applications, the spectrometer may be a reflectance spectrometer of conventional design and may, for example, comprise a fiber optic bundle through which an illuminating source of light passes to irradiate the tissue or cells being analyzed, and through which the light reflected is collected and passed to the spectrometer. The reflected light may be directed through, for example, a collimating slit and subsequently to a diffraction grating. The dispersion of light by the diffraction grating may be intercepted by a linear array of CCDs with suitable sensitivity over the range of wavelengths to be measured. An analog and digital electronic processing device may be used to connect to, or otherwise interface with, a microcomputer supporting the CCD. As previously described, software running on computer system 104 renders the data collected from the CCD array into a graphical plot of intensity versus wavelength of light, such as shown in FIGS. 31 and 32.

The system 28 can also be adapted to evaluate spectral images collected under linear and cross polarized light. Polarization features may be unique with respect to certain tissue pathology, and can thus be compared to differential staining using, for example, thiazine dyes such as methylene blue or toluidine blue O. Single scattered photons can be derived from multi scattered photons at each wavelength, thereby providing an indication of the depth of the stained lesion. In this instance, two linear polarizing filters can be adapted to the system 28. For example, one polarizing filter (not shown) can be placed over the LEDs 6, and another polarizing filter (not shown) can be placed directly in front of the lens 1 of the camera 8. Two sets of spectral images can be taken at each wavelength of interest (e.g., 670 nm and 640 nm for methylene blue), one in a co-linear polarization mode, and the second in a cross polarized mode. The absolute difference between each set can be determined to yield an intensity corresponding to the y-axis of FIGS. 31 and 32. Finally, an analysis using intensity ratios similar to that previously described with regard to FIGS. 31 and 32, can be conducted to differentiate normal cells from, for example, cancerous cells.

In addition to using the system 28, methods in accordance with the present invention can be practiced utilizing other spectrometry devices and components known in the art. For example, an imaging CCD array or a single photocell can be used to record the intensity of light at various wavelengths. In addition, a single electronic photoreceptor, for example, may be used with a combination of filters (as opposed to dispersing the reflected light into a spectrum of light), passing specific wavelengths or bands of wavelengths which may then be compared as a spectrum to obtain plots such as shown in FIGS. 31 and 32. Further, a reflectance photometer and one or more wavelength band pass filters may be used instead of a reflectance spectrometer to obtain the plots such as shown in FIGS. 31 and 32. In each case, an analysis using intensity ratios can be performed to characterize a sample as, for example, normal or abnormal, in a manner as described above.

### Computer Implementation

The techniques of the present invention may be implemented on a computing system 104 such as that depicted in FIG. 33. In this regard, FIG. 33 is an illustration of a computer system 104 which is also capable of implementing some or all of the computer processing in accordance with at least one computer implemented embodiment of the present invention.

Viewed externally, in FIG. 33, a computer system designated by reference numeral 104 has a computer portion 112 having drives 502 and 504, which are merely symbolic of a number of disk drives which might be accommodated by the computer system. Typically, these could include a floppy disk drive 502, a hard disk drive (not shown externally) and a CD ROM 504. The number and type of drives vary, typically with different computer configurations. Disk drives 502 and 504 are in fact optional, and for space considerations, are can be omitted from the computer system used in conjunction with the system 28 and methods described herein.

The computer system 104 also has an optional display monitor 110 upon which visual information pertaining to cells being normal or abnormal, suspected normal, suspected abnormal, etc. can be displayed. In some situations, a keyboard 116 and a mouse 114 are provided as input devices through which input may be provided, thus allowing input to interface with the central processing unit 604. Then again, for enhanced portability, the keyboard 116 can be either a limited function keyboard or omitted in its entirety. In addition, mouse 114 optionally is a touch pad control device, or a track ball device, or even omitted in its entirety as well, and similarly may be used as an input device. In addition, the computer system 104 may also optionally include at least one infrared (or radio) transmitter and/or infrared (or radio) receiver for either transmitting and/or receiving infrared signals.

Although computer system 104 is illustrated having a single processor, a single hard disk drive and a single local memory, the system 104 is optionally suitably equipped with any multitude or combination of processors or storage devices. Computer system 104 is, in point of fact, able to be replaced by, or combined with, any suitable processing system operative in accordance with the principles of the present invention, including hand-held, laptop/notebook, mini, mainframe and super computers, as well as processing system network combinations of the same.

FIG. 34 illustrates a block diagram of the internal hardware of the computer system 104 of FIG. 33. A bus 602 serves as the main information highway interconnecting the other components of the computer system 104. CPU 604 is the central processing unit of the system, performing calculations and logic operations required to execute a program. Read only memory (ROM) 606 and random access memory (RAM) 608 constitute the main memory of the computer system 104. Disk controller 610 interfaces one or more disk drives to the system bus 602. These disk drives are, for example, floppy disk drives such as 502, CD ROM or DVD (digital video disks) drive 504, or internal or external hard drives 614. As indicated previously, these various disk drives and disk controllers are optional devices.

A display interface 618 interfaces display 110 and permits information from the bus 602 to be displayed on the display 110. Again as indicated, display 110 is also an optional accessory. For example, display 110 could be substituted or omitted. Communications with external devices, for example, the other components of the system described herein, occur utilizing communication port 616. For example, optical fibers and/or electrical cables and/or conductors and/or optical communication (e.g., infrared, and the like) and/or wireless communication (e.g., radio frequency (RF), and the like) can be used as the transport medium between the external devices and communication port 616. Peripheral interface 620 interfaces the keyboard 116 and the mouse 114, permitting input data to be transmitted to the bus 602.

In alternate embodiments, the above-identified CPU 604, may be replaced by or combined with any other suitable processing circuits, including programmable logic devices, such as PALs (programmable array logic) and PLAs (programmable logic arrays). DSPs (digital signal processors), FPGAs (field programmable gate arrays), ASICs (application specific integrated circuits), VLSIs (very large scale integrated circuits) and the like.

Any presently available or future developed computer software language and/or hardware components can be employed in such embodiments of the present invention. For example, at least some of the functionality mentioned above could be implemented using Extensible Markup Language (XML), HTML, Visual Basic, C, C++, or any assembly language appropriate in view of the processor(s) being used. It could also be written in an interpretive environment such as Java and transported to multiple destinations to various users.

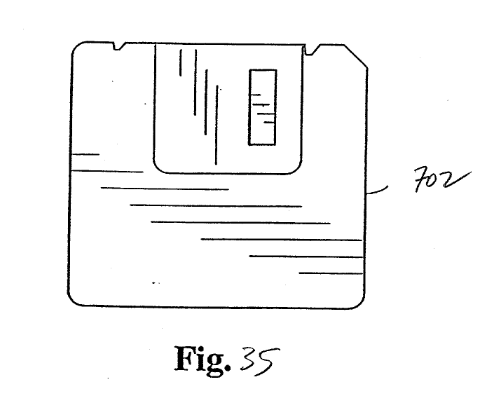

One of the implementations of the invention is as sets of instructions resident in the random access memory 608 of one or more computer systems 104 configured generally as described above. Until required by the computer system 104, the set of instructions may be stored in another computer readable memory, for example, in the hard disk drive 614, or in a removable memory such as an optical disk for eventual use in the CD-ROM 504 or in a floppy disk (e.g., floppy disk 702 of FIG. 35) for eventual use in a floppy disk drive 502. Further, the set of instructions (such as those written in Java, HTML, XML, Standard Generalized Markup Language (SGML), and/or Structured Query Language (SQL)) can be stored in the memory of another computer and transmitted via a transmission medium such as a local area network or a wide area network such as the Internet when desired by the user. One skilled in the art knows that storage or transmission of the computer program medium changes the medium electrically, magnetically, or chemically so that the medium carries computer readable information.

Reference will now be made to specific examples illustrating the bio-spectral imaging system and methods above. It is to be understood that the examples are provided to illustrate preferred embodiments and that no limitation to the scope of the invention is intended thereby.

### EXAMPLE 1

### Diagnosis of Cancer by Hyperspectral Analysis of Methylene Blue Staining of Freshly Excised Human Tissue

### Materials and Methods

Tissue Samples

Patients scheduled for Mohs surgery were enrolled for the use of discard (tumor de-bulking) tissue for metachromatic stain imaging. The imaging system used to analyze the tissue sample is described herein with regard to FIGS. 28-30.

Staining Procedure

Freshly excised bulk discard tissue was pre-rinsed in 1% acetic acid v/v for 30 to 60 seconds using forceps and 60 mm Petri dishes. Maintaining orientation of the cut margin, the tissue was then immersed in either 0.1% or 0.05% Methylene Blue (USP) w/v for 30 to 60 seconds and rinsed again in 1% acetic acid v/v for 30 to 60 seconds. The tissue section was placed on a standard microscope slide with the cut margin facing down. A second slide or cover slip was placed on top of the sample to “hold” or otherwise secure the tissue flat to the lower slide.

The sample was then placed on the stage of the multispectral camera and adjusted for the best field of view (FOV). The 27 band multispectral image set was then acquired under software control and the tissue sample was then processed by conventional histological techniques (cryostat, fixation and automated staining with either toluidine blue O or haematoxylin-eosin) for later examination and comparison of pathology to the spectral image set.

Preliminary Spectral Image Analysis

During this feasibility study, a number of parameters were varied in order to ascertain and evaluate both proper operation of the instrument/software system as well as to determine a preliminary staining protocol. The following image sets are from a sequence of samples where the parameters remained relatively constant.

### Results

Sample 01.025

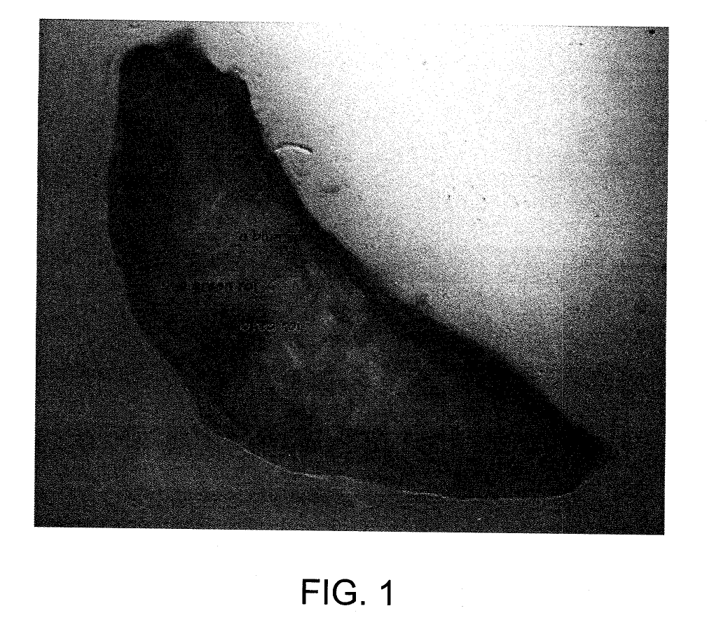

An RGB composite image of sample 01.025 obtained as described above is shown in FIG. 1. Sample 01.025 was taken from a pre surgical biopsy diagnosed basal cell carcinoma (BCC) excision as de-bulked (discard) tissue, immediately pre-rinsed and stained for 60 seconds in 0.05% methylene blue (MB) w/v and post-rinsed. Post surgical histopathology of fixed sections from this specimen were examined and found to be normal tissue with some apparent inflammation. No specific BCC pathology was observed.

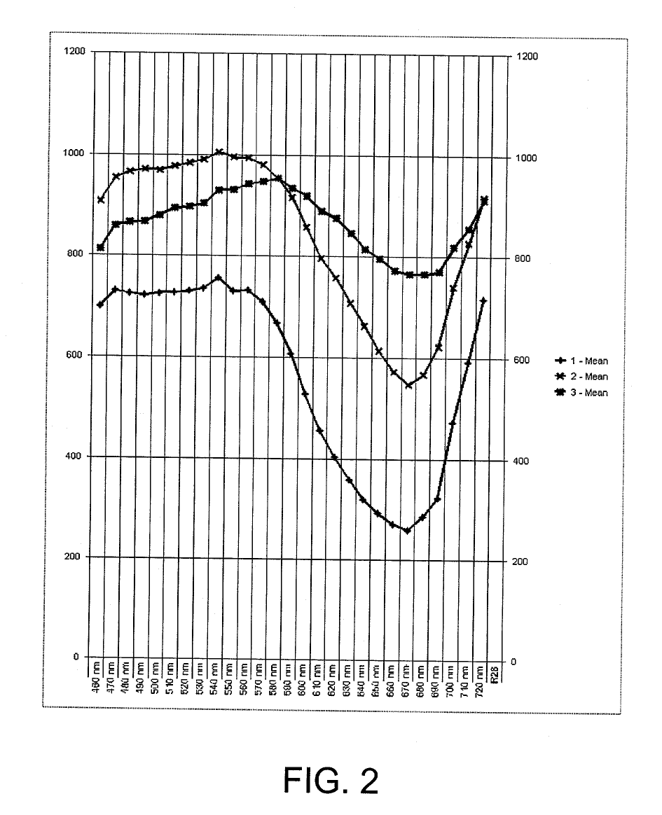

This sample serves as a reference to the properties of methylene blue staining of freshly excised human tissue. Full 27 band spectra were collected and analyzed graphically. Three regions of interest (ROI) were selected here and plotted in the ROI graph 01.025 seen in FIG. 2. The label colors correspond to the spectral selection graph plots.

The most intense apparent staining is graphed as the “red ROI”. As presented graphically, “increasing” absorption of light by the stain “decreases” the recorded light intensity. The Y axis in the graphs represents raw pixel response to light; the lower values corresponding to lower reflected light detected and “greater” absorption. Hence the “peak” absorption is a trough in these graphs. Peak absorption at 670 nm corresponds closely to the peak absorption of aqueous methylene blue at 665 nm. Green and blue ROI's are of correspondingly lower staining intensity. Note the 630 nm feature in the red ROI spectra. The very slight metachromasia suggested here is assigned to “inflammation”.

Sample 01.024.2.2, Set 1

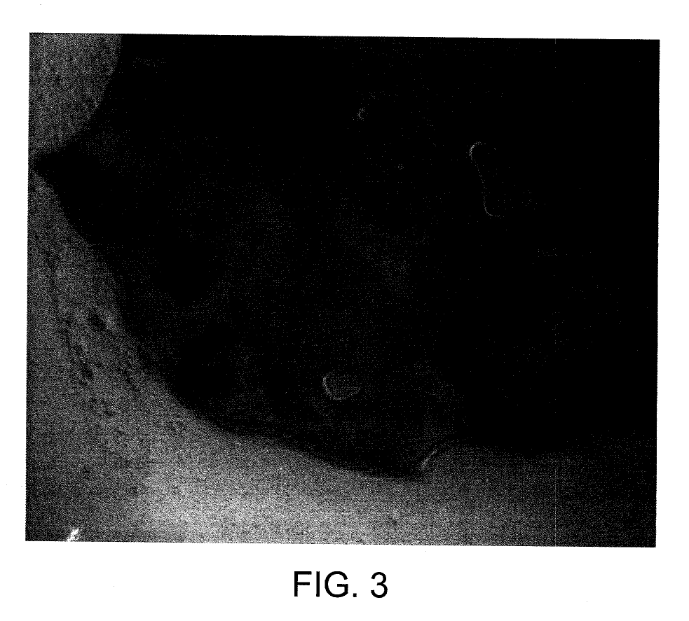

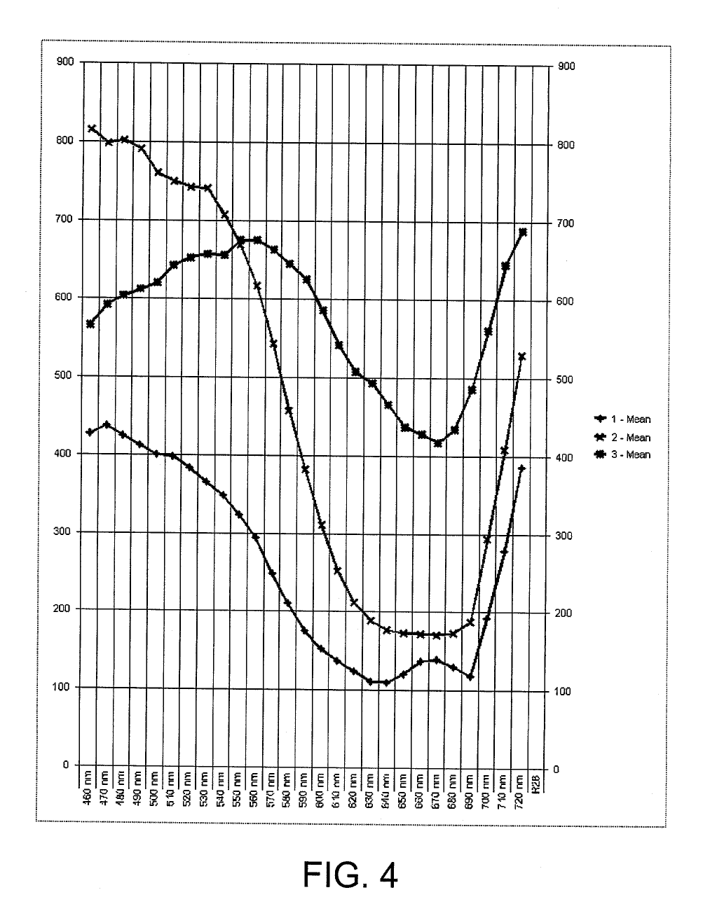

Sample 01.024 was a large section of tissue imaged in a total of 6 sets. Set 1 is shown in FIG. 3 as typical of the entire specimen. Histopathology reveals the “entire section” to be basal cell carcinoma. Because this sample is almost entirely tumor, it serves as a good direct comparison to sample 01.025 as a normal tissue sample. Three “regions of interest” are arbitrarily selected and the corresponding spectral responses are plotted in the graph in FIG. 4.

The “red ROI” in FIG. 4 is probably most typical of an extensive cluster of BCC tumor cells. Note that the spectral regions at 640 nm and 610 nm are of greater relative absorption than the 670 nm band that typically corresponds to methylene blue monomers. Dimerization of methylene blue increases the absorption around 610 nm; increasing polymer aggregates of thiazine stains are known to shift the absorption peak further toward the blue end of the spectrum. These results might suggest that cancer cells preferentially concentrate methylene blue due to the intercellular chemistry and enhanced cell membrane permeability. Dye interaction with the more highly electronegative cell membranes of cancerous and pre cancerous tissue may also shift the absorption spectrum toward the blue.

Sample 01.027

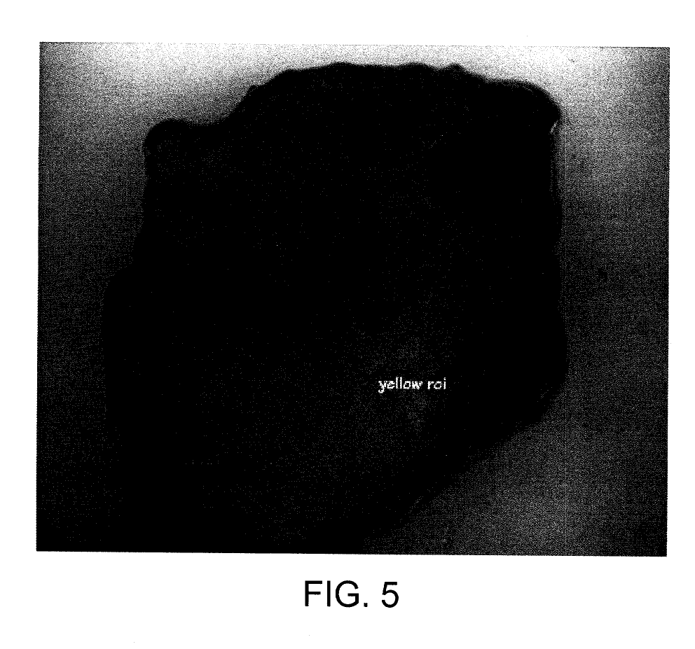

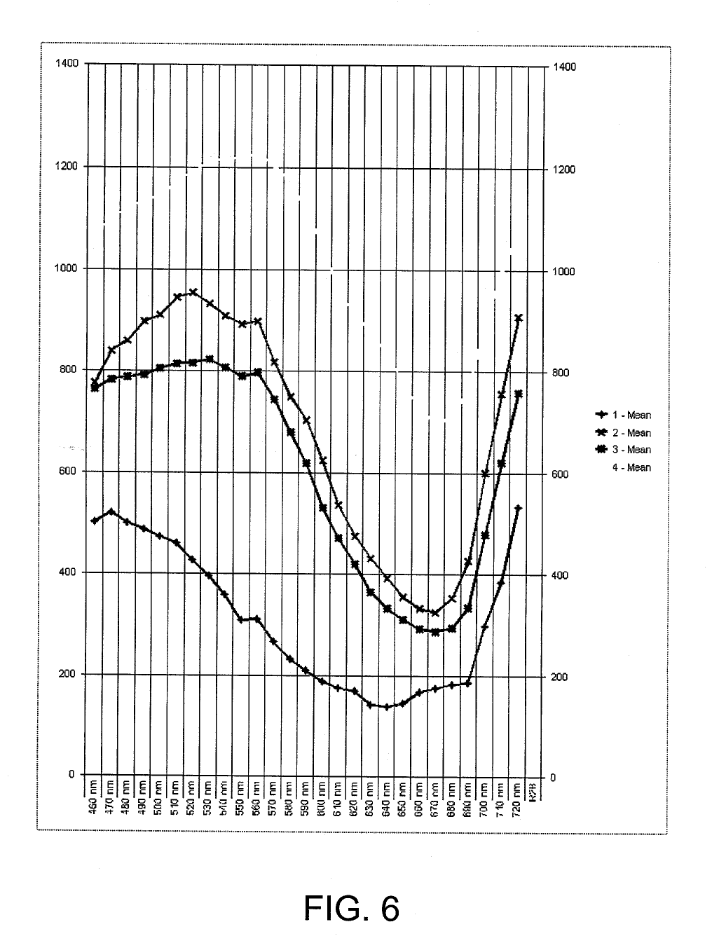

The RGB composite image of sample 01.027 is seen in FIG. 5. Histopathology of sample 01.027 reveals “small regions of basal cell carcinoma with associated inflammation”. Four regions with significant absorption at 670 nm were selected and the corresponding spectra are plotted in FIG. 6. The “red ROI” clearly displays significant metachromatic shift toward the shorter wavelengths comparable to the spectra of the BCC spectra in sample 01.024.2.2. Less significant metachromasia is apparent in the green and blue ROI spectra. The spectra of the yellow ROI would appear more typical of the inflammation seen in sample 01.025, having less absorption in the range 610 nm to 640 nm.

Metaspectral Analysis of Methylene Blue Staining of Freshly Excised Surgical Tissue

From the preliminary evaluation of the multispectral image sets, an empirical determination of key spectral features consistent through the sample sets was made. For those samples displaying a positive metachromatic spectral signature and which correlate with subsequent histopathological diagnosis, the following key features were noted.

The principal absorption spectrum (for methylene blue) centers near 665 to 670 nm and displays a metachromatic shift with an increase in absorption toward the yellow/green wavelengths. By inspection, principal noted features are “shoulders” centered near 600 nm and 640 nm which often exceed the peak absorption intensity at 670 nm. These features correspond to known spectral shifts of methylene blue either resulting from charge-charge association of the dye molecule with electronegative membranes and/or resulting from dimer and oligomer formation of the dye itself. The metachromatic shift resulting from some “active” dye uptake differentiates from normal tissue which characteristically excludes the cationic thiazine dyes (clinical literature). These spectral features are herein defined as metachromatic indices (MI) and the corresponding analysis as “metaspectral” as follows:

MI Class 1: where the relative intensity at 610 nm is equal to or exceeds that at 640 nm and 670 nm respectively.

MI Class 2: where the relative intensity at 640 nm is equal to or exceeds that at 670 nm; 610 nm being less than that at either 640 or 670 nm

MI 01 (600≧610): a sharp shoulder where the relative intensity at 600 nm is equal to or exceeds 610 nm; both bands having less relative intensity than 670 nm.

MI 45 (640≧650): a sharp shoulder where the relative intensity at 640 nm is equal to or exceeds that at 650 nm; both bands having less intensity than 670 nm.

Negative MI (MIØ): no indication of peak broadening to wavelengths shorter than the center wavelength of 670 nm. Metaspectral is defined herein as distinct from hyperspectral. Hyperspectral analysis being based on a spectral band subset determined within the data set based on parameters within the given data set, whereas the spectral band subset for metaspectral analysis is determined by the features of the stain-tissue interaction. “Metaspectral” is defined herein as being a spectral determination of a specific stain with multiple and variable spectral features, where the ratio of the features is determinative of a disease state, and is made using a priori determined specific and discrete wavelengths; either of illumination or of dispersive or diffractive imaged light.

MI 01, MI 45, and MI Class 2 are 2 band analysis referenced to the 670 nm band. MI Class 1 is a 3 band analysis. MI 01 is an implicit subset of MI Class 1 and MI 45 is an implicit subset of MI Class 2. These defined indices form a continuum of metachromasia based spectral signatures that have been correlated with histopathology by empirical means.

Diagnosis of Disease State in Selected Tissue Samples

A subset of samples from 01.001 through 01.027 were used for the creating this “library” of spectral signatures or metachromatic indices. (The principal samples used were 01.019, 024, 025, and 027; these samples being well correlated to the histopathology.) The samples were then tested against the balance of the first set of samples to determine how well the correlation was made. This library of spectral data was then used to diagnose the pathology of tissue samples 01.032-01.040 according to the methods of the present invention. The pathology of samples 01.032-01.040 was also subsequently confirmed by the conventional histological technique described herein.

As seen in Table 1, the diagnosis of the pathology of tissue samples 01.032, 01.038 and 01.040 as non-cancerous and tissue samples 01.033-01.37 and 01.039 as cancerous according to the methods of the present invention agreed with the diagnosis obtained by the conventional technique described above.

TABLE 1
Methylene blue metachromatic index analysis.

| | | | | | Delay | | | | | | | |
|---|---|---|---|---|---|---|---|---|---|---|---|---|
| | | | pretrt/stain | | to | | | MI | MI | | | |
| Sample | | | /destain | | stain | | | class | class | MI | MI | |
| # | type | Stain | sec-sec-sec | location | (min) | Pathology | | 1 | 2 | 01 | 45 | MI 0 |
| 01.003 | bcc | 0.05% MB | 30-30-30 | nose | 5 | Nd | | | + | + | + | |
| | | 0.1% MB | | | 10 | Nd | | + | | + | + | |
| 01.004 | bcc | 0.1% MB | 30-30-30 | nose | 3 | Small tumor sections | + | | | + | + | |
| 01.005 | bcc | 0.1% MB | 30-30-30 | forehead | 2 | Possible tumor, uncertain | +/− | | | + | + | |
| 01.006 | bcc | 0.1% MB | 30-30-30 | shin | 2 | Tangential epidermis, uncertain | +/− | | | + | + | |
| 01.011 | scc inv | 0.05% MB | 30-30-30 | hand | 55 | Nd | | | | | + | |
| 01.013 | Bcc | 0.05% MB /s | 30-30-30 | ear | 16 | Retraction centered tumor | + | | | + | | |
| 01.014 | Bcc | 0.05% MB /s | 30-30-30 | nose | 3 | All tumor | + | | | + | + | |
| 01.015 | scc inv | 0.05% MB /s | 30-30-30 | scalp | 15 | Inflammation, uncertain tu | +/− | + | | + | + | |
| 01.017 | bcc | 0.05% MB | 30-30-30 | scalp | 2 | Negative (All normal) | − | | | | | |
| 01.018 | bcc | 0.05% MB | 30-30-30 | ear | 2 | Half tumor | + | | | + | + | |
| 01.019 | bcc | 0.1% MB | 30-30-30 | cheek | 15 | All tumor | + | | | + | + | |
| 01.020 | bcc | 0.1% MB | 30-30-30 | lip | 11 | Lower half tumor | + | | | | + | |
| 01.021 | bcc | 0.1% MB | 30-30-30 | cheek | 27 | All tumor | + | | | + | + | |
| 01.022 | bcc | 0.1% MB | 30-30-30 | helix | 6 | 1.1 all tumor | + | | | + | + | |
| ″ | | 0.1% MB | | | | 1.2 all tumor | + | | | | + | |
| 01.024 | bcc | 0.05% MB | 60-45-60 | left nasal alar | 5 | 1.1 Mostly tumor; | + | | + | | + | |
| ″ | | | | | | 1.2 mostly tumor | + | | + | | + | |
| 01.025 | bcc | 0.05% MB | 60-60-60 | left nose | 10 | Negative (Normal) | − | | | | | |
| 01.026 | scc in situ | ″ | 60-60-60 | left forehead | 1 | Bowen's sec in situ | + | | + | | + | |
| 01.027 | bcc | ″ | 60-60-60 | left eye | 2 | Sections of tumor, edge & central | + | | + | | + | |
| 01.032 | bcc | ″ | 60-60-60 | rt orbital rim | 7 | Negative | − | | | | | |
| 01.033 | bcc | ″ | 60-60-60 | pre auricular | 2 | Negative/Small BCC* | + | | + | | + | |
| 01.034 | bcc | ″ | 60-60-60 | nose | 2 | Patches of BCC | + | + | + | | | |
| 01.036 | bcc | ″ | 60-60-60 | finger | 2 | Positive on edge | + | | + | | + | |
| 01.037 | bcc | ″ | 60-60-60 | nose | 45 | Distributed BCC buds | + | | + | + | + | |
| 01.038 | bcc | ″ | 60-60-60 | cheek | 35 | Negative (thick section) | − | | | | | |
| 01.039 | bcc | ″ | 60-60-60 | alar | 25 | Multiple BCC sections | + | + | + | + | + | |
| 01.040 | bcc | ″ | 60-60-60 | shin | 3 | Negative | − | | | | | |

Bcc: basal cell carcinoma; Scc: squamous cell carcinoma; Nd: no data (either pathology and/or image data); /s with saline solution; inv: invasiv

### Discussion

Table 1 summarizes the data from twenty six samples and a total of twenty nine separate multispectral image data sets, all using methylene blue and evaluated using a defined set of spectral features (metachromatic indices). All samples were “debulked” tissue sections obtained from a Mohs surgical procedure; the surgical site was determined by prior biopsy and it can be anticipated that most, if not all of the excision samples are positive for either basal or squamous cell carcinoma. Of the 26 samples, 5 samples were found by standard histopathology to be negative.

Using the set of defined metachromatic indices for methylene blue, all samples were evaluated by applying the MI to the multispectral data sets. Significant regions of interest (ROI's) are mapped to the image sets by selection; positive matching of the MI is indicated in Table 1 (one or more of the last five columns).

Initially one false positive was observed with sample 01.033. Microscopic histochemical examination was scored “negative”. The image set scored a moderate MI 45 and MI Class 2 ROI. The corresponding thin section was re-evaluated microscopically; on careful re-examination a small loci of cells typical of basal cell carcinoma was observed at the approximate loci indicated by the image mapping algorithm.

Of a total of 41 tissue sets obtained in this example (some with multiple samples from a given incision), 29 complete sets of data were obtained (and 5 sets for toluidine blue O as described in example 4). Remaining samples were omitted from this comparison because the spectral image data was missing or corresponding histopathology was not obtained (thin sections were not obtained).

With this set of MI's applied to all 29 image sets, no false negative was observed.

### EXAMPLE 2

### Effect of Hemoglobin on the Metachromatic Properties of Methylene Blue Staining of Basal Cell Carcinoma Lesions

Sample 01.024.2.2, Set 2

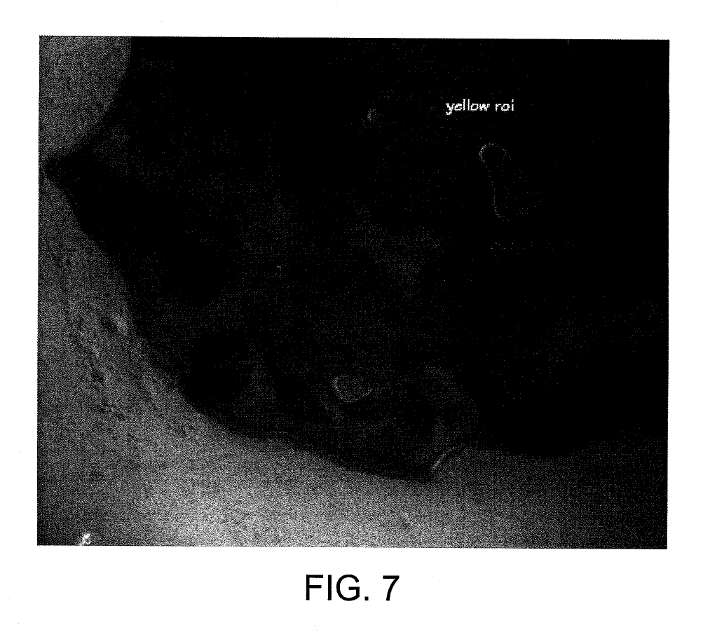

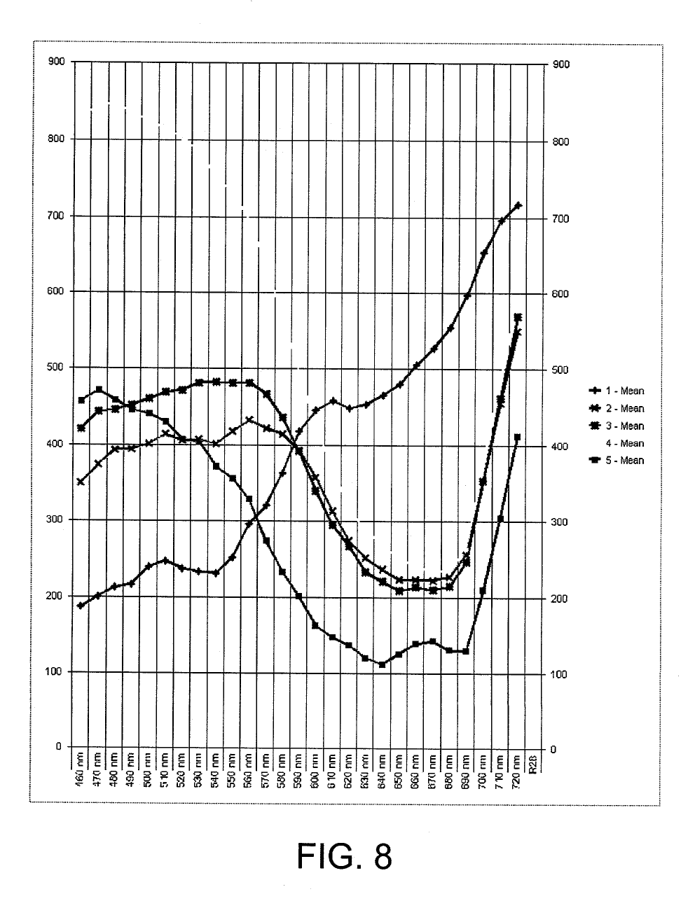

An RGB composite image of another section of sample 01.024.2.2 is shown in FIG. 7. Because this section is clearly BCC, another interesting feature can be examined. This tissue sample was not immediately processed and was allowed to remain in air for approximately 15 minutes. Consequently the residual blood clotted in the tissue and could not be completely removed by pre-rinsing prior to staining. The coagulated hemoglobin can be seen in this image set and was selected for spectral response analysis, plotted in the FIG. 8 together with other ROI's similar to the previous selections. The characteristic hemoglobin spectra can be recognized, despite not being corrected from raw pixel count data. Graphical analysis of potential interference with methylene blue spectra can be made to some extent. This issue is addressed in greater detail herein.

A hemoglobin spectrum is seen in the red ROI plot; little evidence of methylene blue spectra is apparent here. Successive ROI's on shifted methylene blue regions are seen to have a lower absorption in the 460 nm to 560 nm region where heme would have the great absorption. Any interference of heme with the principal metachromatic shift bands would, in this comparison, appear limited.

Image Selectio 01.024 2.2 (Top Region): Heme/Methylene Blue Metachromasia

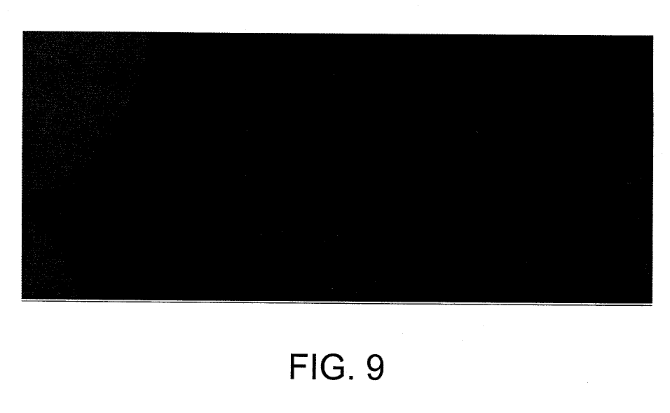

A series of spectral plots was made across the image cube in those regions marked on the RGB composite of image sample 01.024.2.2 seen in FIG. 9. Close inspection shows a sequence going from left to right starting over an area that appears to be primarily heme. The purpose of this spectral sequence is to qualitatively evaluate the potential inference of the heme spectra with that of the metachromatic properties of methylene blue staining of apparent BCC lesions.

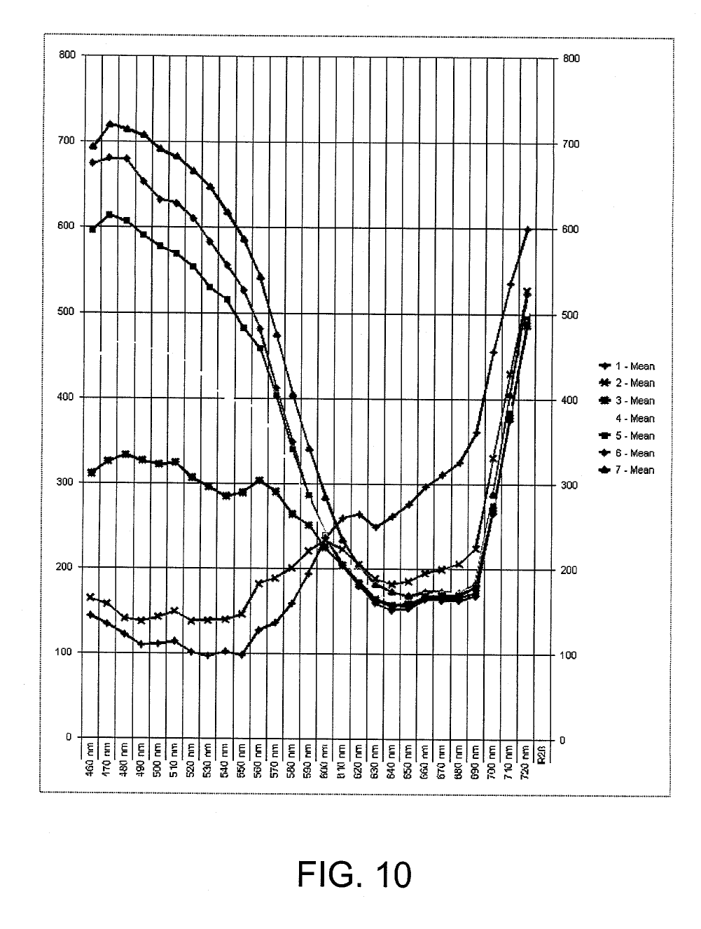

There are seven ROI's selected, from left to right, and plotted in FIG. 10 directly from the image software system.

The red plot corresponds to the left most ROI and appears to be primarily heme with little evidence of methylene blue absorption. Moving right, the next ROI is plotted as green, followed by blue, yellow, magenta, light blue and the right most ROI plotted below as the black graph.

The spectral features observed in the range from 460 nm to 560 nm progressively decrease in absorption, suggesting a decrease in associated heme spectra. The influence of any absorption on the range from 630 nm to 690 nm, the principal regions of methylene blue and associated metachromasia, appears minimal. Note the peak absorption in this series for methylene blue (at roughly 635 nm) remains approximately the same. It would suggest smearing of the blood across the tissue sample in this area that is associated with BCC lesions.

### EXAMPLE 3

### Spectral Mapping of Methylene-blue Stained Tissue Samples-Location of Diseased Tissue

Spectral Mapping of Sample 01.027

The spectra from sample 01.027 were evaluated using a series of 3 band subsets and processed by a feature selection algorithm, in which the spectral features are propagated back through the entire image and then “painted” (false color mapping) on a pixel-by-pixel basis. In this mapping, four subsets based on the relative intensity of the 610, 640 and 670 nm bands were defined and mapped. The spectral mage of sample 01.027 is seen in FIG. 11. Note that these selected colors do not correspond to the spectra plotted in the previous graphs. The red ROI is a “range” selected feature and actually excludes areas displaying “significantly higher” metachromatic properties. However, on close inspection (not readily seen in this image reproduction) the red features are found in isolated clusters on both the lower right edge as well as the central areas—also mapped with feature selection analysis. Those areas mapped in yellow and green are of spectral features displaying less apparent metachromasia than the red ROI; whereas the blue mapped areas are those bordering between inflammation (little metachromasia) and features comparable to known BCC staining.

MI Class Metaspectral Image Mapping

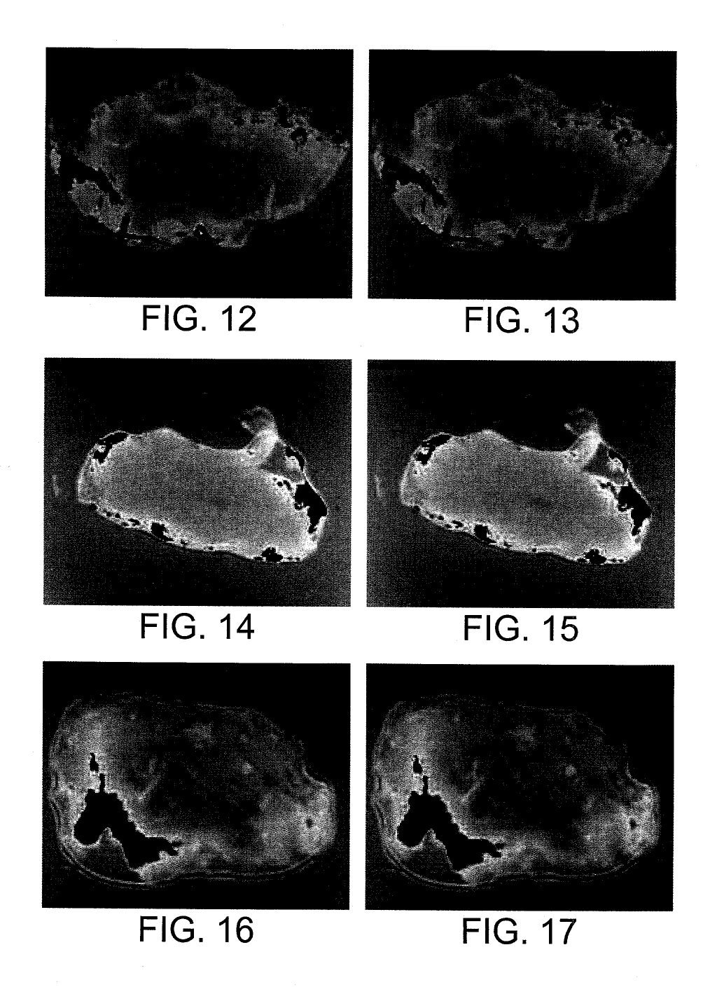

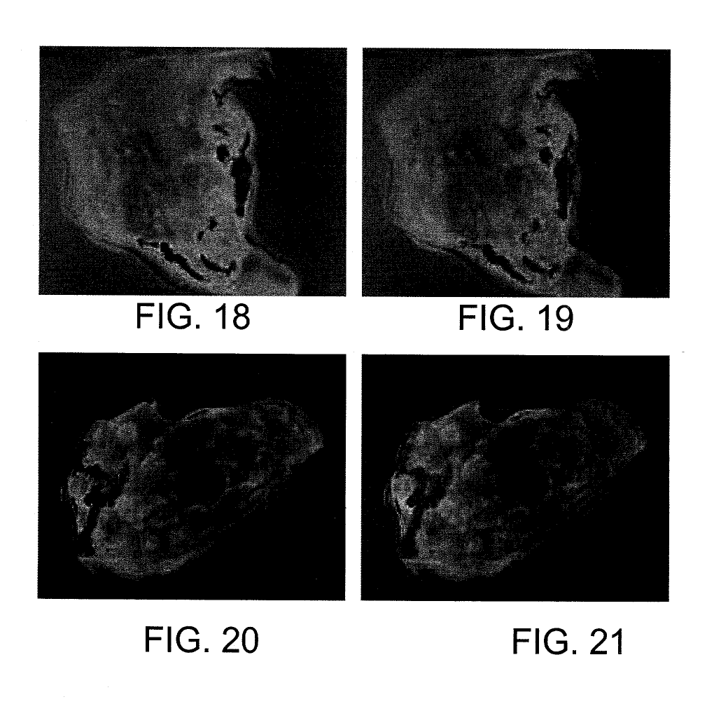

Representative samples of the MI image mapping algorithms defined are provided in FIGS. 12-21. Each photonegative image in the left column is mapped in dark blue corresponding to those voxels matching MI 45 (where the relative intensity at 640 nm equals or exceeds that at 650 nm). The corresponding image in the right column is mapped in light blue together with the MI 45 map and either a MI 01 (where the relative intensity at 600 nm equals or exceeds that at 610 nm) or with MI Class 2 (where the relative intensity at 640 nm equals or exceeds that at 670 nm).

### EXAMPLE 4

### Diagnosis of Cancer by Metaspectral Analysis of Toluidine Blue O Staining of Freshly Excised Surgical Tissue

A similar analysis was performed on a subset of clinical samples (5 samples) using toluidine blue O as the metachromatic stain according to the methods of Example 1, with the exception that toluidine blue O was used at a concentration of 0.1% w/v.

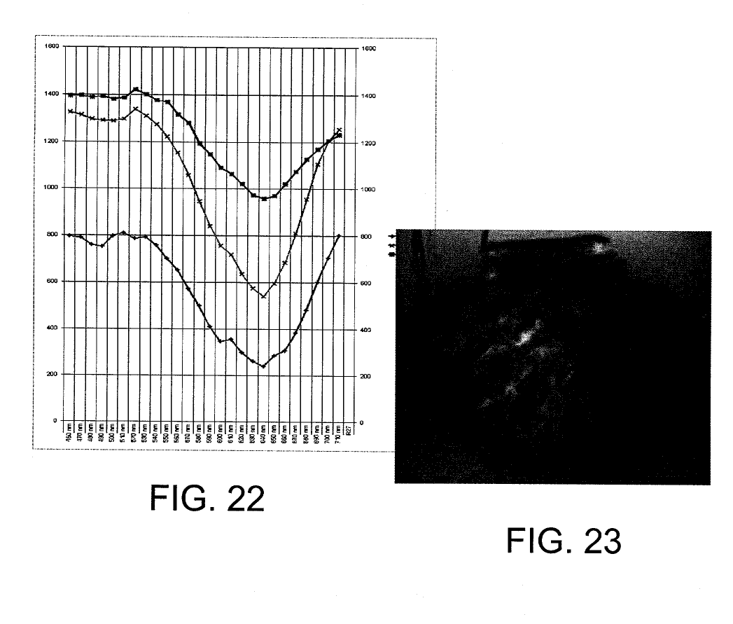

The principal absorption spectrum of toluidine blue O (TBO) centers near 635 to 640 nm and displays a metachromatic shift as an increase in absorption toward the yellow/green wavelengths. By inspection, the principal noted feature is centered near 600 nm (see FIG. 22). In the samples stained with TBO in this present study, the shoulder at 605 nm never exceeded the relative intensity of the main peak at 640 nm. In most areas with relatively limited dye uptake, no distinct shoulder was observed. The metachromatic shift (in this instance the shoulder at 605 nm) resulting from “active” dye uptake differentiates cancerous from normal tissue which characteristically excludes the cationic thiazine dyes (clinical literature-What reference goes here?).

For reference, an RGB color composite of sample 01.008 is presented in FIG. 23 together with corresponding region of interest (ROI) multispectral plots (FIG. 22) corresponding to various areas with differing TBO stain intensity.

The red multispectral plot of FIG. 22 is sampled from the most intense blue stained regions (just right of the center of the tissue sample) seen in FIG. 23; the green is sampled from the perimeter of the region; and the blue is sampled from the more diffuse, lightly blue stained area in the upper left portion of the tissue. Using this feature to define an MI for TBO, the remaining samples were evaluated using the MI 01 feature. The results are presented in Table 2.

As seen in Table 2, tissues determined to be cancerous by a conventional technique were correctly identified as cancerous tissue samples when practicing the method according to the present invention.

TABLE 2
Toluidine blue O metachromatic index analysis.

| | | | pretrt/stain | | Delay | | | | | | |
|---|---|---|---|---|---|---|---|---|---|---|---|
| | | | /destain | | to | | | | | | |
| | | | sec-sec- | | stain | | MI class | MI class | | | |
| Sample# | type | Stain | sec | location | (min) | Pathology | 1 | 2 | MI 01 | MI 45 | MI 0 |

| | | | | | | | | |
|---|---|---|---|---|---|---|---|---|
| 01.007 | bcc | 0.1% TBO | 30-30-30 | cheek | 5 | 1.1 All tumor in center | + | + |
| | | | | | | (circular sebaceous gland) | | |
| ″ | | | | | | 1.2 | + | + |
| 01.008 | bcc | 0.1% TBO | 30-30-30 | scalp | 3 | Uncertain small section | + | + |
| | | | | | | marked | | |
| 01.009 | scc | 0.1% TBO | 30-30-30 | temple/ear | 20 | retraction, certain tumor; | + | + |
| | invasive | | | | | | | |
| 01.010 | bcc | 0.1% TBO | 30-30-30 | forehead | 25 | Nd | | + |
| 01.023 | bcc | 0.1% | 30-30-30 | nose | 5 | Nd | | |
| | | TBO/a&e | | | | | | |

Bcc: basal cell carcinoma; Scc: squamous cell carcinoma; Nd: no data (either pathology and/or image data); a&e: 1% acetic acid & 5% ethanol

### EXAMPLE 5

### Identification of the Location of Diseased Tissue by Spectral Mapping of Toluidine Blue O Stained Tissue Samples

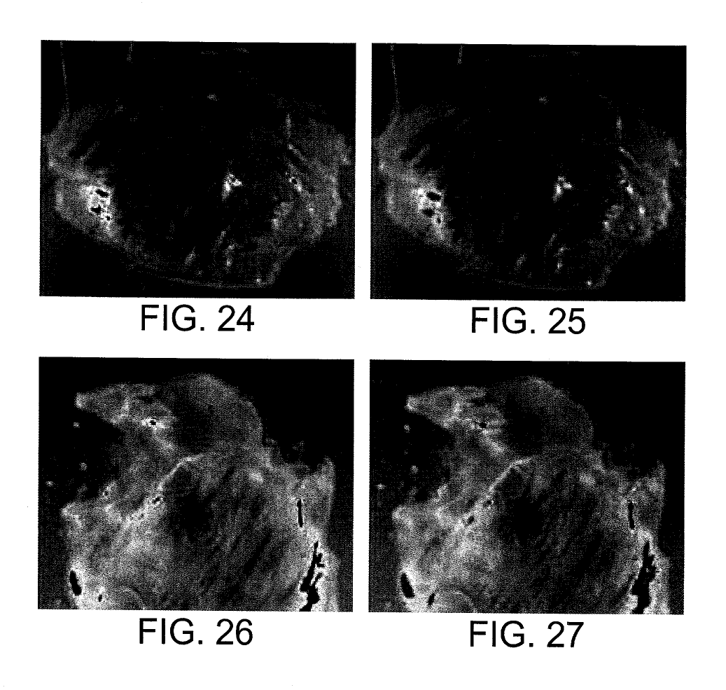

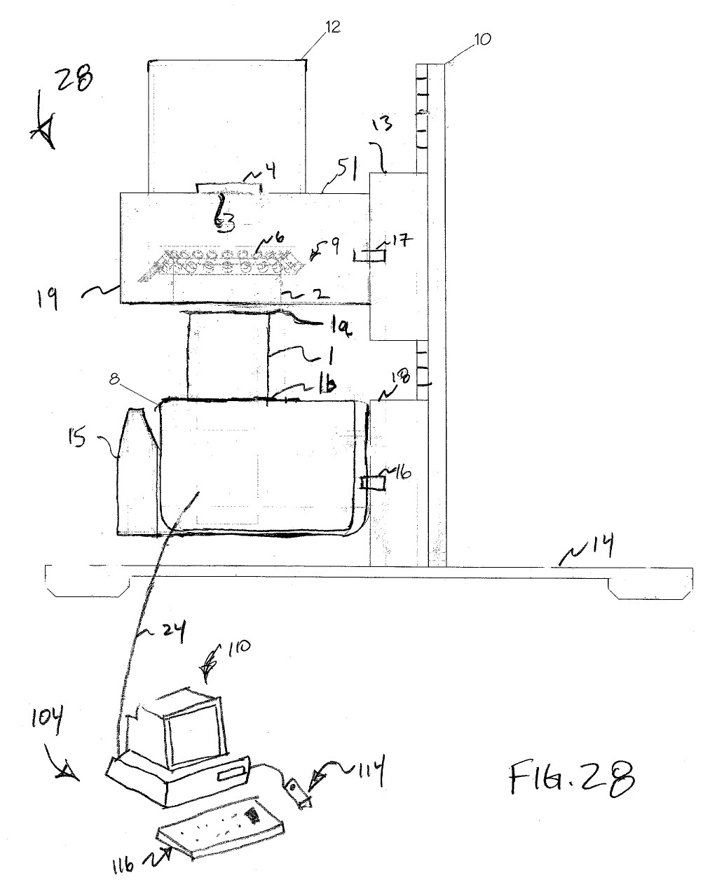

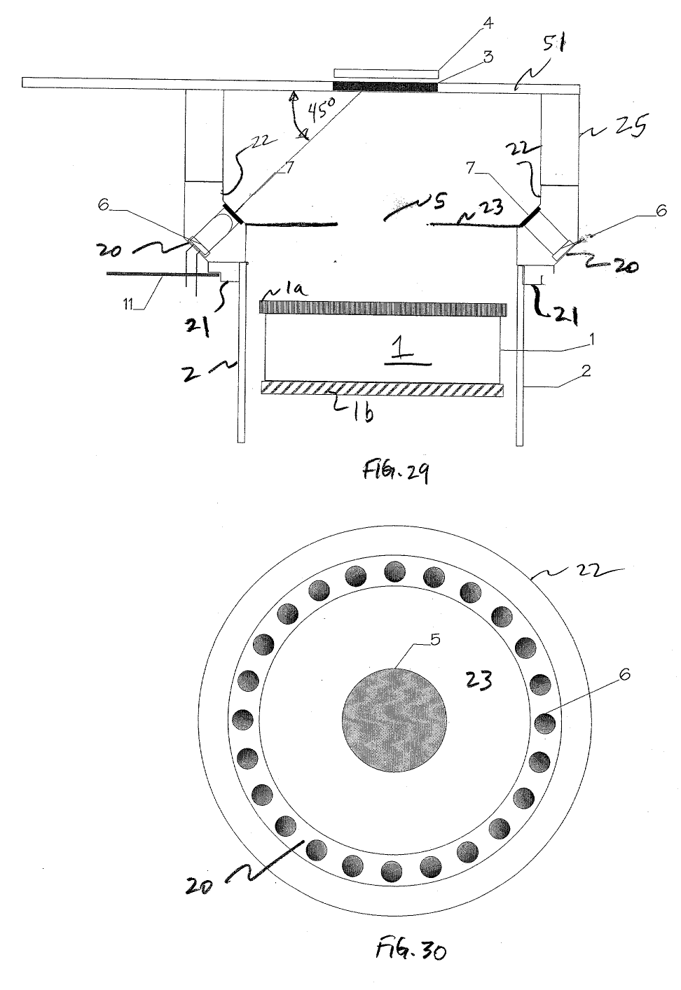

MI Class metaspectral image mapping: representative samples of the MI image mapping algorithms defined for Toluidine blue O (TBO) are shown for two of the samples in FIGS. 24-27. Each photonegative image in the left column is mapped in dark blue corresponding to those voxels matching MI 01 (for TBO, equivalent to that derived for methylene blue) (where the relative intensity at 600 nm equals or exceeds that at 610 nm). The corresponding image in the right column is mapped in light blue together with the MI 01>640 map where the 600/610 nm absorption intensity is greater than that at 640 nm.

### EXAMPLE 6

### Diagnosis of Dysplasia or Carcinoma in the Oral Cavity or Cervix

In this example, spectral image analysis is used to correlate the metachromatic properties of toluidine blue O with the presence of dysplasia or carcinoma in the oral cavity or cervix. This analysis can be performed in a matter of minutes and provides a presumptive determination of the presence of dysplasia or carcinoma, such as squamous cell carcinoma, before a patient leaves the examining room. Stained cells are metaspectrally imaged on a membrane at two or more wavelengths coincident with the peak absorption spectra of the monomeric and oligomeric forms of the stain. The images are analyzed on the basis of 1) the presence or absence of thiazine dye in the cells and 2) the ratio of monomer to oligomer retained by the cells as determined by examining the extent of the metachromatic shift of the dye from a reflected light spectrum of the stained tissue or cells with the extent of the metachromatic shift of the dye from a library of previously obtained spectra of similarly stained cells as described herein. The frequency of epithelial cells stained by the thiazine dye, combined with the ratio of monomer to oligomer found in the cells, determine the potential presence of dysplastic, pre-cancerous and cancerous cells. Metraspectral imaging can reproducibly detect subtle degrees of metachromasia by measuring both monomers and oligomers in the same cells.

A standard cervical or oral cavity cytology specimen, taken with a brush designed for such purposes and readily available, or obtained by fine needle aspirations utilizing an appropriate instrument known to the art, is transferred to a 15 ml disposable test tube containing 0.01% toluidine blue O in 2% acetic acid/5% ethanol. The specimen material is shaken and dispersed in this medium for 60 to 90 seconds and then is removed from the test tube and aspirated through a 25 mm diameter 5 micron microporous nitrocellulose filtration membrane. The filtration removes the extraneous and dispersed mucosal debris and retains intact epithelial cells. The aspiration distributes the retained epithelial cells across the full diameter of the membrane. The retained cells are then rinsed on the membrane by slowly aspirating through the membrane 50 ml of 2% acetic acid 5% ethanol. This rinse step destains any non-specifically stained epithelial cells and associated mucosal debris that remain on the membrane. The membrane holder is then opened and a 25 mm optical window placed directly over the retained cells on the surface of the membrane. The membrane/optical window is placed on the stage of a spectral imager and immediately analyzed.

The spectral imager may be a commercial Nikon D1X Digital camera with an array size of 23.7 mm by 15.6 mm having 4024 pixels by 1324 pixels (5.47 megapixel CCD). The D1X is further coupled with preferably a 60 mm F2.8 micro Nikkor lens in close focus configuration having a primary magnification of 1:1. This provides a direct pixel resolution of approximately 6-11 microns so that a typical epithelial cell would have at least 3 pixels that record absorption at the imaged wavelengths. A smaller range of resolution (e.g., 7-9 microns per pixel) could also be utilized. For analysis using the stain toluidine blue O, the spectral imager takes a series of three two-dimensional digital images of the surface of the membrane and all retained epithelial cells at each of the following metaspectral wavelengths: 640 nm, 600 nm and 573 nm.

Three (or more—as may be desirable to have available for other metaspectral wavelengths) opposing pairs of high luminosity light emitting diodes (LED's) are mounted in a ring attached to and around the front perimeter of the 60 mm micro Nikkor lens and are oriented with a beam direction 45 degrees to the orthogonal of the optical axis and the plane of the membrane/optical window stage. Uniform dispersion of the light beam from the LED's is preferably obtained via a holographic diffuser in front of the emitted beam. Above the diode ring, and integral to it, is an imaging stage that accepts the membrane/optical window assembly in the focal plane of the camera lens. Uniform illumination of the membrane is accomplished by the LED/diffuser pairs at the imaging stage. Final focus, if necessary, is achieved by integral focusing of the lens/D1X camera combination.

At an adjusted exposure commensurate with the available illumination provided by the respective LED pairs, a grayscale monochrome digital image is acquired at each of the three specified metaspectral wavelength bands in rapid sequence. The digital image arrays are then transferred to a computer for subsequent analysis and display.

The method of manipulating the arrays to derive determinate data may include, for example, flat field calibration and response normalization.

An entire grayscale monochrome image array acquired at 640 nm is scanned for groups of 3 or more adjacent pixels (pixel clusters) having an intensity corresponding to moderate to significant retention by cells of toluidine blue O. If more than three adjacent pixels are found, they are subclassified as one or more cells based on the total number of adjacent pixels and their relative spatial orientation, together with the integrated intensity value. Thus, a total count of toluidine blue O stained epithelial cells is made. These counts are then verified by comparison to the same algorithm performed on images acquired at 600 nm and 573 nm to confirm the presence and number of stained cells in the field.

The pixel clusters are further classified by determining the ratio of the intensities at 573 nm and 600 nm to that at 640 nm. Each three band voxel cluster (a voxel is defined as a stack of pixels, each pixel representing a different spectral band) is then assigned a metachromatic index based on these intensity ratios and placed in one of a series of metaspectral index classifications as shown in the following table.

| | Imaged cells/ | Index | |
|---|---|---|---|
| | Imaged | level | Suggestive indication |
| | voxel sets | value | (cervical specimen) |
| Total | 573/420 | | |
| 640/600/573 index | 573/420 | n/a | Inflammation or non- |
| 1 ·• 0.2 ·• | | | specific |
| 0.2 | | | |
| 640/600/573 index | 73/71 | Level 1: .13 | Atypical squamous |
| 1 ·• 0.5 ·• | | | cells of undetermined |
| 0.5 | | | significance |
| 640/600/573 index | 3/1 | Level 2: .02 | Squamous |
| 1 ·• 0.8 ·• | | | intraepithelial lesion |
| 0.5 | | | |
| 640/600/573 index | 0 | Level 3: 0.0 | Cervical intraepithelial |
| 1 ·• 1.0 ·• | | | neoplasia |
| 0.8 | | | |
| 640/600/573 index | 1/1 | Level 4: .02 | Cancer |
| 1 ·• 1.0 ·• | | | |
| 1.0 | | | |

This table is computed and the number of cells at each “index level value” displayed along side an image of the cells on the membrane. The image allows the operator to evaluate the adequacy of the specimen. Further, visual indication of those voxels that report an elevated index are highlighted in some significant fashion, such as false color, on the monitor. The values shown in the table are based on preliminary studies and may be refined with additional clinical studies to further correlate the metachromatic index to conventional cytological analysis.

The results are reported within several minutes of taking the specimen. The presence of a highly indexed cell (showing significant metachromatic shift) would indicate the presence of a lesion requiring further evaluation, such as colposcopy (in application to the cervix) and biopsy, which could be performed at the same visit.

One advantage of the above method over the conventional PAP smear is that the determination of pre-cancerous and cancerous cells is made by direct spectral cytochemical analysis rather than subjective screening for aberrant morphology by a human cytologist, where a certain percentage of positive smears are missed, or by automated slide readers that use spatial algorithms to search for aberrant morphology.

Methylene blue may replace toluidine blue O directly in the above example. If methylene blue is used, the metaspectral wavelengths used would be 665 nm (monomer), 635 nm and 610 nm. The LED's would change to correspond to these principle absorption bands. Additional correlation of the metaspectral index to clinical results would also be required. The spectral characteristics of methylene blue could allow the use of only two bands, for example 665 and 609 nm to effectively measure and correlate the metachromatic index, further simplifying and reducing the cost of the associated instrumentation. The use of three or more bands for any given stain may give a more detailed metaspectral analysis for use in metachromatic index determination. Other members of the thiazine class, such as the azures and thionine could be used with similar modifications.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method for in situ diagnosis of a disease state in biological tissue or cells of a living organism, comprising:
a) applying to test tissue or cells in situ a biological stain composition containing at least one metachromatic dye to form stained test tissue or cells;
b) obtaining at least one reflected light spectrum of the stained test tissue or cells;
c) determining the degree of the metachromatic shift of the dye from the reflected light spectrum of the stained test tissue or cells;
d) comparing the degree of the metachromatic shift of the dye from the reflected light spectrum of the stained test tissue or cells with the degree of the metachromatic shift of the dye from a library of previously obtained spectra of similarly stained reference tissue or cells, said similarly stained reference tissue or cells having been previously categorized as diseased or non-diseased; and
e) correlating the reflected light spectrum of the stained test tissue or cells with a disease state, whereby an in situ diagnosis of a disease state is made.

2. The method of claim 1, wherein the metachromatic dye is a thiazine dye.

3. The method of claim 2, wherein the metachromatic dye is toluidine blue O, methylene blue or a combination thereof.

4. The method of claim 2, wherein the tissue or cells are epithelial cells.

5. The method of claim 1, wherein the spectra of the similarly stained tissue or cells are contained in a database and the step of comparing is performed by a microprocessor.

6. The method of claim 1, wherein said obtaining at least one reflected light spectrum includes illuminating the stained test tissue or cells with light and directing the reflected light to a spectrometer.

7. The method of claim 6, wherein the spectrometer measures light within the range of wavelengths from between about 200 to 1100 nanometers.

8. The method of claim 1, wherein the spectra of the stained test tissue or cells are obtained using a photometer and one or more light filters.

9. The method of claim 1, wherein said stained reference tissue or cells have been previously categorized as diseased or non-diseased by a histological technique.

10. The method of claim 1, wherein the tissues or cells are selected from the group consisting of skin, cervix, vagina, mouth, colon, and esophagus.

11. The method of claim 1, further comprising obtaining one or more spectra on the test tissues or cells prior to staining the test tissue or cells and subtracting said one or more spectra of the unstained test tissues or cells from the one or more spectra of the stained test tissue or cells prior to comparing the spectra of the stained test tissue or cells with the spectra of the similarly stained reference tissue or cells.

12. The method of claim 1, further comprising rinsing the stained test tissue or cells prior to obtaining the spectra on the stained tissue or cells.

13. The method of claim 1, wherein the disease state is pre-cancer or cancer.

14. The method of claim 1, wherein the disease state is caused by a microorganism selected from the group consisting of bacteria, fungi and viruses.

15. A method for in situ diagnosis of a disease state in biological tissue or cells of a living organism, comprising:
(a) creating a library of spectra from reference tissue or cells stained with a biological stain composition including at least one metachromatic dye to form stained reference tissue or cells, said stained reference tissues or cells previously categorized as diseased or non-diseased;
(b) applying to test tissue or cells a biological stain composition including at least one metachromatic dye to form stained test tissue or cells;
(c) performing a spectral analysis of the stained test tissue or cells to obtain at least one spectrum;
(d) comparing the spectrum from the stained test tissue or cells with the spectra from the library of spectra of the reference tissue or cells; and
(e) correlating the spectrum of the stained test tissue or cells with a disease state, whereby an in situ diagnosis of a disease state is made.

16. The method of claim 15, wherein said histological technique includes sectioning and freezing the reference tissue or cells and staining the reference tissue or cells with a biological stain composition.

17. The method of claim 15, wherein said metachromatic dye is a thiazine dye.

18. The method of claim 17, wherein said thiazine dye is methylene blue, toluidine blue O or a combination thereof.

19. The method of claim 15, wherein said creating a library of spectra includes obtaining reflected light spectra of said stained reference tissue or cells.

20. The method of claim 15, wherein said performing a spectral analysis includes obtaining a reflected light spectrum of said stained test tissue or cells.

21. The method of claim 15, wherein said disease state is pre-cancer or cancer, or is a disease state caused by a microorganism selected from the group consisting of bacteria, fungi and viruses.

22. The method of claim 15, wherein said reference tissue or cells have been categorized as diseased or non-diseased by a histological technique.

23. A method for in situ diagnosis of a disease state in biological tissue or cells of a living organism, comprising:
(a) comparing the degree of the metachromatic shift of a metachromatic dye from a reflected light spectrum of said tissue or cells stained with said dye with the degree of the metachromatic shift of the dye from a library of spectra of similarly stained tissue or cells, said similarly stained tissue or cells previously categorized as diseased or non-diseased; and
(b) correlating the reflected light spectrum with a disease state, whereby an in situ diagnosis of a disease state is made.

24. A method of creating a library of spectra of biological tissue or cells of a living organism, comprising:
(a) applying to the tissue or cells a biological stain composition including at least one metachromatic dye to form stained tissue or cells;
(b) determining whether said stained tissue or cells are diseased or non-diseased;
(c) performing a spectral analysis of the stained tissue or cells to obtain at least one spectrum;
(d) analyzing the spectral features of said spectrum; and
(e) correlating said spectral features to said disease state.

25. The method of claim 24 wherein said metachromatic dye is a thiazine dye.

26. The method of claim 25, wherein said thiazine dye is methylene blue, toluidine blue O, or a combination thereof.

27. The method of claim 24, wherein said spectral features include the degree of the metachromatic shift of the stain.

28. The method of claim 24, wherein said spectrum is a reflected light spectrum.

29. The method of claim 24, wherein said determining whether said stained tissue or cells are diseased or non-diseased is performed by a histological technique.

30. A method for monitoring the efficiency of photodynamic therapy of diseased biological tissue or cells in vivo, comprising:
(a) applying to test tissue or cells a phototosensitizing metachromatic dye to form stained test tissue or cells;
(b) obtaining a reflected light spectrum of the stained test tissue or cells;
(c) determining the extent of the metachromatic shift of the dye from the reflected light spectrum of the stained test tissue or cells;
(d) comparing the extent of the metachromatic shift of the dye from the reflected light spectrum of the stained test tissue or cells with the extent of the metachromatic shift of the dye from reflected light spectra of similarly stained reference tissue or cells that have been previously categorized as diseased or non-diseased;
(e) irradiating said stained test tissue or cells with light having an intensity sufficient to induce photooxidation of said dye; and
(f) monitoring the changes in the degree of the metachromatic shift of the dye by repeating (b), (c) and (d).

31. The method of claim 30, wherein said dye is a thiazine dye.

32. The method of claim 31, wherein said thiazine dye is methylene blue, toluidine blue O or a combination thereof.

33. The method of claim 30, wherein said similarly stained reference tissue or cells have been previously categorized as diseased or non-diseased by a histological technique.

34. A method for monitoring the efficiency of photodynamic therapy of diseased biological tissue or cells in vivo, comprising:
(a) applying to said tissue or cells a photosensitizing metachromatic dye to form stained test tissue or cells;
(b) performing a spectral analysis on said stained test tissue or cells to obtain at least one spectrum;
(c) comparing said at least one spectrum of said stained test tissue or cells to a library of previously obtained spectra of similarly stained reference tissue or cells, said similarly stained reference tissue or cells previously categorized as diseased or non-diseased;
(d) irradiating said stained test tissue or cells with light having an intensity sufficient to induce photooxidation of said dye; and
(e) monitoring the changes in the spectrum of said stained test tissue or cells by repeating (b) and (c).

35. The method of claim 34, wherein said metachromatic dye is a thiazine dye.

36. The method of claim 35, wherein said thiazine dye is methylene blue, toluidine blue O, or a combination thereof.

37. The method of claim 34, wherein said spectrum is a reflected light spectrum.

38. The method of claim 37, wherein said comparing said at least one spectrum of said stained test tissue or cells to a library of previously obtained spectra of similarly stained reference tissue or cells includes comparing the degree of the metachromatic shift of the dye from the reflected light spectrum of said stained test tissue or cells with the degree of the metachromatic shift of the dye from the spectra of said similarly stained reference tissue or cells.

39. A method for improving the efficiency of photodynamic therapy of diseased biological tissue or cells in vivo, comprising:
(a) applying to said tissue or cells a phototosensitizing metachromatic dye to form stained test tissue or cells;
(b) obtaining a reflected light spectrum of the stained test tissue or cells;
(c) determining the extent of the metachromatic shift of the dye of the reflected light spectrum;
(d) comparing the extent of the metachromatic shift of the dye from the reflected light spectrum of the stained test tissue or cells with the extent of the metachromatic shift of the dye from reflected light spectra of similarly stained reference tissue or cells that have been previously categorized as diseased or non-diseased;
(e) irradiating said stained test tissue or cells with light having an intensity sufficient to induce photooxidation of said dye;
(f) monitoring the changes in the metachromatic shift of the dye by repeating (b), (c) and (d); and
(g) adjusting the intensity of the irradiating light or duration of the therapy, repositioning the source of the irradiating light, or a combination thereof, based on the difference between the extent of the metachromatic shift of the dye from the reflected light spectrum of the stained test tissue or cells with the extent of the metachromatic shift of the dye from the reflected light spectrum of said similarly stained reference tissue or cells.

40. The method of claim 39, wherein the spectrometer measures light within the range of wavelengths from about 200 nanometers to about 1100 nanometers.

41. The method of claim 39, wherein the reflected spectrum is measured and recorded using a photometer and at least one light filter.

42. The method of claim 39, wherein the tissues or cells are selected from the group consisting of skin, cervix, vagina, mouth, colon and esophagus.

43. The method of claim 39, wherein said tissue or cells are epithelial cells.

44. A bio-spectral imaging system comprising:
at least two light sources having a substantially same first wavelength;
at least two light sources having a substantially same second wavelength, the second wavelength being different than the first wavelength;
an imaging plane, adapted to support a plurality of stained cells positioned adjacent said imaging plane, receiving sequential illumination from at least two of said at least two light sources at the first wavelength and from at least two of said at least two light sources at the second wavelength; and
a plurality of photoreceptors, arranged for movement relative to said imaging plane, positioned to digitally record an image of at least a portion of the stained cells, during each of the sequential illuminations of the cells at the first and second wavelengths.

45. The system according to claim 44, further comprising a processor for comparing the metachromatic shift, at the first and second wavelengths, of at least a portion of data corresponding to the image.

46. The system according to claim 45, wherein the processor categorizes, based on the metachromatic shift, at least a portion of the cells as having normal cell characteristics.

47. The system according to claim 45, wherein the processor categorizes, based on the metachromatic shift, at least a portion of the cells as having abnormal cell characteristics.

48. The system according to claim 47, wherein the categorization is abnormal when the stain reflects no more light at the second wavelength approximately 30 nanometers less that the first wavelength at which minimum reflection of light occurs in normal cells.

49. The system according to claim 48, wherein the first wavelength is approximately 665 nanometers.

50. The system according to claim 48, wherein the first wavelength is approximately 638 nanometers.

51. The system according to claim 48, wherein the first wavelength is approximately 602 nanometers.

52. The system according to claim 44, wherein the light sources are concentrically spaced around said plurality of photoreceptors.

53. The system according to claim 52, wherein the light sources have an angle of incidence of approximately 45 degrees with respect to said imaging plane.

54. The system according to claim 44, wherein each of said light sources having a first wavelength are spaced substantially equidistant with respect to each other.

55. The system according to claim 44, wherein each of said light sources having a second wavelength are spaced substantially equidistant with respect to each other.

56. The system according to claim 44, further comprising a holographic diffuser associated with each light source and arranged to receive light therefrom.

57. A bio-spectral imaging system comprising:
at least two light sources having a substantially same first wavelength;
at least two light sources having a substantially same second wavelength, the second wavelength being different than the first wavelength;
a plurality of photoreceptors positioned to digitally record an image, during each of the sequential illuminations of the cells at the first and second wavelengths; and
an imaging plane, arranged for movement relative to said plurality of photoreceptors, adapted to support a plurality of stained cells positioned adjacent said imaging plane, receiving sequential illumination from at least two of said at least two light sources at the first wavelength and from at least two of said at least two light sources at the second wavelength.

58. The system according to claim 57, further comprising a processor for comparing the metachromatic shift, at the first and second wavelengths, of at least a portion of data corresponding to the image.

59. The system according to claim 58, wherein the processor categorizes, based on the metachromatic shift, at least a portion of the cells as having normal cell characteristics.

60. The system according to claim 58, wherein the processor categorizes, based on the metachromatic shift, at least a portion of the cells as having abnormal cell characteristics.

61. The system according to claim 60, wherein the categorization is abnormal when the stain reflects no more light at the second wavelength approximately 30 nanometers less that the first wavelength at which maximum reflection of light occurs in normal cells.

62. The system according to claim 61, wherein the first wavelength is approximately 665 nm.

63. The system according to claim 61, wherein the first wavelengths is approximately 638 nm.

64. The system according to claim 61, wherein the first wavelength is approximately 602 nm.

65. The system according to claim 57, wherein the light sources are concentrically spaced around said plurality of photoreceptors.

66. The system according to claim 65, wherein the light sources have an angle of incidence of approximately 45 degrees with respect to said imaging plane.

67. The system according to claim 57, wherein said light sources comprise light emitting diodes.

68. The system according to claim 57, further comprising a holographic diffuser arranged to receive light from each of said light sources.

69. The system according to claim 57, wherein each of said light sources having a first wavelength are spaced substantially equidistant with respect to each other.

70. The system according to claim 57, wherein each of said light sources having a second wavelength are spaced substantially equidistant with respect to each other.

71. The system according to claim 57, further comprising a holographic diffuser associated with each light source and arranged to receive light therefrom.

72. A bio-spectral imaging system comprising:
an imaging plane, adapted to support a plurality of stained cells positioned adjacent said imaging plane, receiving sequential illumination from at least two light sources having a substantially same first wavelength and from at least two light sources having a substantially same second wavelength, the second wavelength being different than the first wavelength;
a first platform, arranged for movement relative to said imaging plane, capable of carrying a digital camera having a lens positioned to record a digital image of at least a portion of the plurality of stained cells; and
a second platform, positioned between said imaging plane and said first platform, having a plurality of apertures for receiving a plurality of light sources, the apertures being positioned at an angle of approximately 45 degrees with respect to said imaging plane.

73. The system according to claim 72, further comprising a processor for comparing the metachromatic shift, at the first and second wavelengths, of at least a portion of data corresponding to the image.

74. The system according to claim 73, wherein the processor categorizes, based on the metachromatic shift, at least a portion of the cells as having normal cell characteristics.

75. The system according to claim 74, wherein the processor categorizes, based on the metachromatic shift, at least a portion of the cells as having abnormal cell characteristics.

76. The system according to claim 75, wherein the categorization is abnormal when the stain reflects no more light at the second wavelength approximately 30 nanometers less that the first wavelength at which minimum reflection of light occurs in normal cells.

77. The system according to claim 76, wherein the first wavelength is approximately 665 nanometers.

78. The system according to claim 76, wherein the first wavelength is approximately 638 nanometers.

79. The system according to claim 76, wherein the first wavelength is approximately 602 nanometers.

80. The system according to claim 73, wherein the light sources are concentrically spaced around a lens of a camera positioned to capture an image of at least a portion of the stained cells.

81. The system according to claim 80, wherein the light sources have an angle of incidence of approximately 45 degrees with respect to said imaging plane.

82. The system according to claim 71, wherein each of said light sources having a first wavelength are spaced substantially equidistant with respect to each other.

83. The system according to claim 71, wherein each of said light sources having a second wavelength are spaced substantially equidistant with respect to each other.

84. The system according to claim 71, further comprising a holographic diffuser arranged to receive light from at least one of the light sources.

85. A computer program medium storing computer instructions therein for instructing a computer to perform a computer-implemented process for categorizing stained tissue or cells of a living organism as having at least one of diseased characteristics and non-diseased characteristics, the medium comprising:
a) computer program code means for determining the degree of metachromatic shift of the stain from a reflected light spectrum of the stained test tissue or cells;
b) computer program code means for comparing the degree of the metachromatic shift of the stain from the reflected light spectrum of the stained test tissue or cells with the degree of the metachromatic shift of the stain from previously obtained spectra of similarly stained reference tissue or cells, said similarly stained reference tissue or cells having been previously categorized as diseased or non-diseased; and
c) computer program code means for correlating the reflected light spectrum of the stained test tissue or cells with a disease state.

86. The computer program medium according to claim 85, wherein the reflected light spectrum is within the range of wavelengths from between about 500 to 700 nanometers.

87. The computer program code of claim 85, wherein said disease state is selected from the group consisting of dysplasia, pre-cancer and cancer.

88. A computer program medium storing computer instructions therein for instructing a computer to perform a computer-implemented process for categorizing stained tissue or cells of a living organism as having at least one of diseased characteristics and non-diseased characteristics, the medium comprising:
computer program code means for comparing reflected light spectrum from the stained test tissue or cells with a substantially same reflected light spectrum from stained reference tissue or cells previously categorized as diseased or non-diseased; and
computer program code mean for correlating the spectrum of the stained test tissue or cells with a disease state.

89. The computer program medium according to claim 88, wherein the reflected light spectrum is within the range of wavelengths from between about 500 to 700 nanometers.

90. The computer program code of claim 88, wherein said disease state is selected from the group consisting of dysplasia, pre-cancer and cancer.

91. A computer program medium storing computer instructions therein for instructing a computer to perform a computer-implemented process for categorizing stained tissue or cells of a living organism as having at least one of diseased characteristics and non-diseased characteristics, the medium comprising:
computer program code means for comparing the degree of the metachromatic shift of a metachromatic stain from a reflected light spectrum of the stained tissue or cells with the degree of the metachromatic shift of the stain from a predetermined spectra of similarly stained tissue or cells, said similarly stained tissue or cells previously categorized as diseased or non-diseased; and
computer program code means correlating the reflected light spectrum with a disease state.

92. The computer program medium according to claim 91, wherein the reflected light spectrum is within the range of wavelengths from between about 500 to 700 nanometers.

93. The computer program code of claim 91, wherein said disease state is selected from the group consisting of dysplasia, pre-cancer and cancer.

94. A bio-spectral imaging system comprising:
at least one light source having a substantially same first wavelength;
at least one light source having a substantially same second wavelength, the second wavelength being different than the first wavelength;
an imaging plane, adapted to support a plurality of stained cells positioned adjacent said imaging plane, receiving sequential illumination from at least one of said at least one light source at the first wavelength and from at least one of said at least one light source at the second wavelength; and
a plurality of photoreceptors, arranged for movement relative to said imaging plane, positioned to digitally record an image of at least a portion of the stained cells, during each of the sequential illuminations of the cells at the first and second wavelengths.

95. A bio-spectral imaging system comprising:
at least one light source having a substantially same first wavelength;
at least one light source having a substantially same second wavelength, the second wavelength being different than the first wavelength;
a plurality of photoreceptors positioned to digitally record an image, during each of the sequential illuminations of the cells at the first and second wavelengths; and
an imaging plane, arranged for movement relative to said plurality of photoreceptors, adapted to support a plurality of stained cells positioned adjacent said imaging plane, receiving sequential illumination from at least one of said at least one light source at the first wavelength and from at least one of said at least one light source at the second wavelength.

96. A bio-spectral imaging system comprising:
an imaging plane, adapted to support a plurality of stained cells positioned adjacent said imaging plane, receiving sequential illumination from at least one light source having a substantially same first wavelength and from at least one light source having a substantially same second wavelength;
a first platform capable of carrying a digital camera having a lens positioned to record a digital image of at least a portion of the plurality of stained cells, arranged for movement relative to said imaging plane; and
a second platform, positioned between said imaging plane and said first platform, having a plurality of apertures for receiving said at least one light source having the substantially same first wavelength and said at least one light source having the substantially same second wavelength, at least a portion of said second platform having an angle of incidence with respect to said imaging plane of approximately 45 degrees.

97. A bio-spectral imaging system comprising:
an imaging plane, adapted to support a plurality of stained cells positioned adjacent said imaging plane, receiving sequential illumination from at least two light sources having a first wavelength and from at least two light sources having a second wavelength, the second wavelength being different than the first wavelength;
a plurality of photoreceptors, arranged for movement relative to said imaging plane, positioned to digitally record an image of at least a portion of the stained cells, during each of the sequential illuminations of the cells at the first and second wavelengths; and
a platform disposed between said imaging plane and said photoreceptors for carrying at least two light sources having a substantially same first wavelength and at least two light sources having a substantially same second wavelength, the photoreceptors having an angle of incidence of approximately 45 degrees with respect to said imaging plane.

98. A bio-spectral imaging system comprising:
a plurality of photoreceptors positioned to digitally record an image, during each of sequential illuminations of the cells at first and second wavelengths, the second wavelength being different than the first wavelength;
an imaging plane, arranged for movement relative to said plurality of photoreceptors, adapted to support a plurality of stained cells positioned adjacent said imaging plane, receiving sequential illumination from at least two light sources at the first wavelength and from at least two light sources at the second wavelength; and
a platform disposed between said imaging plane and said photoreceptors for carrying the at least two light sources at the first wavelength and the at least two light sources at the second wavelength, the light sources having an angle of incidence of approximately 45 degrees with respect to said imaging plane.

99. A bio-spectral imaging system comprising:
an imaging plane, adapted to support a plurality of stained cells positioned adjacent said imaging plane;
a first platform capable of carrying a digital camera having a lens positioned to record a digital image of at least a portion of the plurality of stained cells, arranged for movement relative to said imaging plane; and
a second platform, positioned between said imaging plane and said first platform, having a plurality of apertures for receiving at least two light sources having the substantially same first wavelength and at least two light sources having the substantially same second wavelength, the second wavelength being different than the first wavelength, the apertures having an angle of approximately 45 degrees with respect to said imaging plane.

100. A bio-spectral imaging system comprising:
at least two light sources having a substantially same first wavelength;
at least two light sources having a substantially same second wavelength, the second wavelength being different than the first wavelength;
an imaging plane, adapted to support a plurality of stained cells positioned adjacent said imaging plane, receiving sequential illumination from at least two of said at least two light sources at the first wavelength and from at least two of said at least two light sources at the second wavelength; and
means for capturing an image of at least a portion of the stained cells, arranged for movement relative to said imaging plane, during each of the sequential illuminations of the cells at the first and second wavelengths.

101. A bio-spectral imaging system comprising:
at least two light sources having a substantially same first wavelength;
at least two light sources having a substantially same second wavelength, the second wavelength being different than the first wavelength;
an imaging plane, arranged for movement relative to said plurality of photoreceptors, adapted to support a plurality of stained cells positioned adjacent said imaging plane, receiving sequential illumination from at least two of said at least two light sources at the first wavelength and from at least two of said at least two light sources at the second wavelength; and
means for capturing an image of at least a portion of the stained cells during each of the sequential illuminations of the cells at the first and second wavelengths.
